(19) United States
(12) Patent Application Publication
(10) Pub. No.: US 2025/0090550 A1
(43) Pub. Date: Mar. 20, 2025

(54) INJECTABLE COMPOSITION COMPRISING CYTOLYTIC COMPOUND IN GEL, GEL-FORMING SOLUTION OR GEL-FORMING SUSPENSION FOR REDUCTION OF FAT

(71) Applicant: GLONOVA PHARMA CO., LTD., Taipei City (TW)

(72) Inventors: Minhsiung KAO, New Taipei, (TW); Yongyu CHEW, Hsinchu (TW)

(73) Assignee: GLONOVA PHARMA CO., LTD., Taipei City (TW)

(21) Appl. No.: 18/565,564

(22) PCT Filed: Jul. 5, 2022
(86) PCT No.: PCT/CN2022/103787

§ 371 Date: Nov. 30, 2023

(51) Int. Cl. A61K 31/573; A61K 8/42; A61K 8/43; A61K 8/63; A61K 9/00; A61K 31/155; A61K 31/167

(52) U.S. Cl. CPC A61K 31/573; A61K 8/42; A61K 8/43; A61K 8/63; A61K 9/0019; A61K 31/155; A61K 31/167; A61K 2800/91

(57) ABSTRACT

The present invention provides an injectable composition comprising cytolytic compound, preferably deoxycholic acid or a salt thereof, more preferably DCA-Na as a first component; and a pharmaceutically acceptable excipient. It also provides use of the injectable composition, for the reduction or removal of localized fat in a subject in need thereof, wherein the injectable composition is subcutaneously injected into a subcutaneous injection site of the subject. It also provides a method for reducing or removing localized fat in a subject in need thereof, comprising administering to the subject, an effective amount of the injectable composition. In particular, the injectable composition of the invention may be in the form of gel.

### CROSS-REFERENCE TO RELATED APPLICATIONS

This application claims priority claim under 35 U.S.C. § 119 (a) on PCT/CN2022/103787 filed 5 Jul. 2022, the entire contents of which are incorporated herein by reference.

### FIELD OF THE INVENTION

The present invention relates to preparation of injectable compositions. More particularly, the present invention relates to an injectable composition comprising cytolytic compound in gel, gel-forming solution or gel-forming suspension for reduction of fat; use or method for the reduction or removal of localized fat by administering the injectable composition of the invention. In particular, the injectable composition of the invention may be in the form of gel during or after injection.

### BACKGROUND OF THE INVENTION

Submental fat or double chin is usually resistant to diet or exercise, therefore the non-surgical fat removal injection with the active ingredient deoxycholic acid has become a novel treatment to reduce submental fat.

Deoxycholic acid (DCA) is a secondary bile acid which can emulsify and solubilize fat for digestion and absorption in the intestine. Its salt, sodium deoxycholate (DCA-Na), is an anionic detergent commonly used to lyse cells. DCA is a TGR5 agonist (Takeda G-protein-coupled receptor 5, GPBAR1) and the activation of TGR5 was found to reduce obesity in high-fat-diet fed animals. DCA is predicted to lyse adipocytes and resulting in fat reduction. However, cytolysis will attract inflammatory cells such as macrophages and monocytes to remove destroyed fat cells. Patients who received deoxycholic acid treatment will commonly experience swelling, pain, numbness, redness, and areas of hardness in the treatment due to the inflammation, and thus the interval between each treatment is long (around a month) as the histological evidence showed that posttreatment inflammation was largely resolved by this time. DCA-Na can form hydrogels under low pH, mixing with tris(hydroxymethyl)aminomethane (TRIS) buffer or mixing with polymers and an amino acid, L-aspartic acid. The release of additional solutes on the DCA-Na/TRIS hydrogels was found to be sustained, thus should be a suitable drug deliver and release platform. Although a study showed that adding with amino acids L-lysine and L-arginine, but not glycine and L-α-alanine, weakened their hydrogel formation, we successfully constructed a DCA-Na gel system by mixing with basic amino acids, such as L-lysine, L-arginine and L-histidine, and/or organic acid, such as acetic acid.

Studies have shown that, after injection of deoxycholic acid solution, deoxycholic acid permeates into fat tissue more than 1 centimeter. A fat tissue ball with diameter more than 2-centimeter goes into inflammation reaction. When deoxycholate gel solution is injected into fat tissue, only fat cells surrounding deoxycholate gel are destroyed gradually during 7-days slow-releasing of deoxycholate. Inflammation reaction is limited to this less than 2 millimeters thin layer of fat cells surrounding deoxycholate gel. Total volume of inflammatory fat tissue is less than 10% of traditional cytolytic injection. Finally, a cavity with volume proportional to injected dose of deoxycholate appears in fat tissue, and disappears within 2˜3 weeks.

Thus, a slow-releasing deoxycholic acid, or its salt sodium deoxycholate (DCA-Na) gel at the injected sites is expected, which is constructed by mixing with basic amino acids, such as L-lysine, L-arginine and L-histidine, and/or organic acid, such as acetic acid, so that the cytolytic reaction could be limited to deoxycholate-immersed fat cells surround gel surface. Anti-inflammatory drug or local anesthetic could also be added to the injections during the treatment to reduce inflammation and pain. Moreover, we also aimed to increase the concentration of DCA-Na so that cytolysis could be more effective, thus patients can complete their treatment within fewer treatment sessions. Taken together, the mixture of DCA-Na, basic amino acid and/or organic acid, and anti-inflammatory drug and/or local anesthetic should reduce or remove fat, and effectively reduce the adverse effects, and reduce the interval between each treatment and the whole treatment process. The compositions of DCA-Na injections will preferably form a gel-like appearance later than 5 minutes and before 120 minutes after mixing.

### SUMMARY OF THE INVENTION

The present invention provides an injectable composition of cytolytic compound, preferably deoxycholic acid or a salt thereof, more preferably DCA-Na, in the form of gel, gel-forming solution or gel-forming suspension. The injectable composition may be used for reducing or removing localized fat, and have less adverse effects and relatively short treatment process.

In one aspect, the invention provides an injectable composition comprising cytolytic compound in gel, gel-forming solution or gel-forming suspension for reduction of fat, comprising: a cytolytic compound as a first component; and a pharmaceutically acceptable excipient.

Preferably, the cytolytic compound is deoxycholic acid or a salt thereof.

More preferably, the cytolytic compound is DCA-Na, and the injectable composition further comprises a second component selected from one or more of a basic amino acid or an organic acid.

In some embodiments, the concentration of DCA-Na is 7-51 mg/mL.

In some embodiments, the basic amino acid is L-lysine.

In one embodiment, the concentration of L-lysine is 11-145 mg/mL.

In another embodiment, the pH of L-lysine before mixing is <8.0, and the pH of the injectable composition is 6.45-7.75.

In another embodiment, the injectable composition further comprises an anti-inflammatory drug as a third component.

Preferably, the anti-inflammatory drug is aspirin.

More preferably, the concentration of aspirin is 14-100 mg/mL.

Preferably, the injectable composition further comprises a local anesthetic as a fourth component.

More preferably, the local anesthetic is Lidocaine.

More preferably, the concentration of Lidocaine is 2.5-6.5 mg/mL.

Preferably, the anti-inflammatory drug is Dexamethasone Sodium Phosphate (DSP).

More preferably, the pH of the injectable composition is 6.45-7.40.

More preferably, the concentration of DSP is not more than 1 mg/mL.

In some embodiments, the basic amino acid is L-histidine.

Preferably, the concentration of L-histidine is 1.4-11.5 mg/mL.

In some embodiments, the basic amino acid is L-arginine.

Preferably, the concentration of L-arginine is 115-143 mg/mL. In some embodiments, the organic acid is acetic acid.

Preferably, the concentration of acetic acid is 46-143×10⁻³%.

In other embodiments, the injectable composition further comprises saline.

In other embodiments, the injectable composition is in the form of a gel, preferably during and after injection.

In another aspect, the invention provides use of the injectable composition described above, for the reduction or removal of localized fat in a subject in need thereof, wherein the injectable composition is subcutaneously injected into a subcutaneous injection site of the subject.

In another embodiment, the subcutaneous injection site is the localized fat within face, chin, arm, waist, abdomen or thigh of the subject.

In another aspect, the invention provides use of the injectable composition described above, for production of a medicine for the reduction or removal of localized fat.

In another aspect, the invention provides a method for reducing or removing localized fat in a subject in need thereof, comprising administering, preferably subcutaneously injecting to the subject, an effective amount of the injectable composition described above.

In another embodiment, the subject is human.

In another embodiment, the injectable composition is administered, preferably subcutaneously injecting to the localized fat within face, chin, arm, waist, abdomen or thigh of the subject.

The injectable composition of the invention may also comprise saline, and may be in the form of gel during or after injection.

### BRIEF DESCRIPTION OF THE DRAWINGS

FIG. 1 Appearances of the mixture of DCA-Na solutions and (a) 100 mg/mL, (b) 200 mg/mL, (c) 300 mg/mL, (d) 400 mg/mL or (e) 500 mg/mL L-lysine solutions.

FIG. 2 Appearances of the mixture of DCA-Na solutions and (a) 200 mg/mL or (b) 400 mg/mL L-lysine solutions in various pH.

FIG. 3 Appearances of the mixture of DCA-Na solutions and (a) 90 mg/mL, (b) 180 mg/mL, (c) 300 mg/mL, (d) 450 mg/mL or (e) 600 mg/mL LA solutions.

FIG. 4 Appearances of the mixture of DCA-Na solutions and (a) 90 mg/mL, (b) 180 mg/mL, (c) 300 mg/mL, (d) 450 mg/mL or (c) 600 mg/mL LA in Lidocaine HCl solutions.

FIG. 5 Photos of fat tissues collected at both sides (L: left side, R: right side) of the 2 pigs, wherein (a) and (b) are from the first pig, and (c) and (d) are from the second pig.

FIG. 6 Appearances of the mixture of DCA-Na solutions and (a) 200 mg/mL or (b) 400 mg/mL L-lysine/DSP solutions in various pH.

FIG. 7 Photos of fat tissues collected at both sides (L: left side, R: right side) of the 3 pigs, wherein (a) and (b) are from the first pig, (c) and (d) are from the second pig, and (e) and (f) are from the third pig.

FIG. 8 Appearances of the mixture of DCA-Na solutions and (a) 2.5 mg/mL, (b) 5 mg/mL, (c) 10 mg/mL, (d) 20 mg/mL, (c) 40 mg/mL or (f) 50 mg/mL L-histidine solutions.

FIG. 9 Appearances of the mixture of DCA-Na solutions and 500 mg/mL L-arginine solutions.

FIG. 10 Appearances of the mixture of DCA-Na solutions and (a) 0.1%, (b) 0.2%, (c) 0.3%, (d) 0.4%, (e) 0.5%, or (f) 0.6% L-histidine solutions.

### DETAILED DESCRIPTION OF PREFERRED EMBODIMENTS

### Definitions

In the invention, the following definitions are applicable:

The articles “a” and “an” are used in this invention to refer to one or more than one (i.e., to at least one) of the grammatical object of the article. By way of example, “an element” means one element or more than one element.

The term “and/or” is used in this invention to mean either “and” or “or” unless indicated otherwise.

The term “effective amount” means an amount of a composition according to the invention which, in the context of which it is administered or used, is sufficient to achieve the desired effect or result. An effective amount can be determined by methods known to those of skill in the art.

A “subject” is a mammal, e.g., a human, mouse, rat, guinea pig, dog, cat, horse, cow, pig, or non-human primate, such as a monkey, chimpanzee, baboon or rhesus. Subject of the invention is preferably a human.

A “pharmaceutically acceptable excipient” may be used herein, and refers to a compound that is useful in preparing a pharmaceutical composition that is generally safe, non-toxic and neither biologically nor otherwise undesirable, and includes excipients that are acceptable for veterinary use or human pharmaceutical use. A pharmaceutically acceptable excipient as used in the specification and claims includes both one and more than one such excipient. Suitable excipients include: solvents, such as sterile water or water for injection; lubricating agents such as talc, magnesium stearate; wetting agents; emulsifying and suspending agents; tonicity agent, such as sodium chloride; acid, such as hydrochloric acid; base, such as sodium hydroxide; buffer, such as dibasic sodium phosphate; and preserving agents such as methyl- and propylhydroxy-benzoates and benzyl alcohol.

A “cytolytic compound” may also be a detergent or a lipolytic compound. Suitable cytolytic compounds include, but are not limited to phosphatidylcholine, deoxycholic acid or a salt thereof. Cytolytic compound of the invention is preferably deoxycholic acid or a salt thereof, more preferably DCA-Na.

Aspirin (acetylsalicylic acid) is a nonsteroidal anti-inflammatory drug (NSAID) used to reduce pain, fever, or inflammations but also suppresses the normal functioning of platelets. Its soluble salt lysine aspirin (LA) can be administered intravenously or intramuscularly. After administration, lysine aspirin is converted into acetylsalicylic acid and metabolized into salicylic acid.

Dexamethasone is a glucocorticosteroid similar to a natural hormone produced by adrenal glands. It relieves inflammation (swelling, heat, redness, and pain) and is used to treat certain forms of arthritis, severe allergies, asthma and certain types of cancer. Dexamethasone sodium phosphate (DSP) is its sodium phosphate salt form.

Lidocaine (or lignocaine) is a local anesthetic of the amino amide type which can temporarily blocks transmission of nerve impulses. It typically begins working within several minutes and lasts for half an hour to three hours after administered. Lidocaine mixtures may also be applied directly to the skin or mucous membranes to numb the area.

### EXAMPLES

The present invention can be better understood according to the following examples. However, it would be easy for a person skilled in the art to understand that the contents described in the examples are merely intended to illustrate the present invention rather than limit the present invention described in detail in the claims. Unless otherwise indicated, compositions of the present invention can be prepared by using commercially available materials and utilizing general techniques and procedures known to those skilled in the art.

### DCA-Na Solutions

DCA-Na (99%, Acros Organics, Geel, Belgium), NaOH, Na2HPO4 (Sigma-Aldrich, St. Louis, MO, USA) and NaCl (Honeywell, Charlotte, NC, USA) were added to 80 mL water for injection and then made up to 100 mL solution. Benzyl alcohol (Alfa Aesar, Ward Hill, MA, USA) was then added to the solution and additional sodium hydroxide/hydrochloric acid was added to adjust the pH value. The amounts and concentrations of various ingredients were as shown in Tables 1 and 2 to prepare 5% and 1% solutions respectively. Solutions were sterilized by autoclave for 30 minutes.

TABLE 1

| | Amount | Concentration |
|---|---|---|
| Ingredients | (mg) | (mg/mL) |
| Sodium deoxycholate (DCA-Na) | 5280 | 52.8 |
| Dibasic sodium phosphate | 142 | 1.42 |
| (Na₂HPO₄) | | |
| Sodium chloride (NaCl) | 438 | 4.38 |
| Benzyl alcohol | 900 | 9 mg |
| Water for injection | Up to 100 | — |
| | mL | |

5% solution: 52.8 mg/mL DCA-Na (equivalent to 50 mg/mL DCA, 100 mL, pH 8.3)

TABLE 2

| | Amount | Concentration |
|---|---|---|
| Ingredients | (mg) | (mg/mL) |
| Sodium deoxycholate (DCA-Na) | 1056 | 10.56 |
| Sodium hydroxide (NaOH) | 40 | 0.4 |
| Dibasic sodium phosphate | 142 | 1.42 |
| (Na₂HPO₄) | | |
| Sodium chloride (NaCl) | 438 | 4.38 |
| Benzyl alcohol | 900 | 9.00 |
| Water for injection | Up to 100 | — |
| | mL | |

1% solution: 10.56 mg/mL DCA-Na solution (equivalent to 10 mg/mL DCA, 100 mL, pH 8.3)

In the following examples, DCA-Na solutions were mixed with other components to prepare an injectable composition. Unless otherwise stated, the requirements for the final concentration of DCA-Na in the obtained compositions were ≥70% of initial solutions (≥36.96 mg/mL for 5% solution, ≥7.39 mg/mL for 1% solution). The appearances after mixing DCA-Na with other components were observed after placing at 25, 37 and 42° C. for 20, 30, 45, 60 and 120 minutes. 200 μL of the mixtures were also added to 200 μL 0.9% saline respectively and their appearances were also observed after placing at 37° C. for 20, 30, 45, 60 and 120 minutes. Photos were taken and showing in the figures.

### Example 1. Compositions of Dca-Na and L-Lysine

To test if compositions of DCA-Na and L-lysine form gel after mixing, DCA-Na solutions were mixed with acidic L-lysine solutions (pH 5.0-5.2, Acros Organics) according to TABLE 3.

TABLE 3

| Group | 1 | 2 | 3 | 4 | 5 |
|---|---|---|---|---|---|
| L-Lysine (g) | 1.0 | 1.0 | 1.0 | 1.0 | 1.0 |
| ddH2O (mL) | 10.0 | 5.0 | 3.3 | 2.5 | 2.0 |
| Final concentration of | 100 | 200 | 300 | 400 | 500 |
| L-lysine (mg/mL) | | | | | |
| Group | 1-1 | 1-2 | 1-3 | 1-4 | 1-5 |
| 5% DCA-Na solution | 1.00 | 1.00 | 1.00 | 1.00 | 1.00 |
| (mL) | | | | | |
| 100 mg/mL L-lysine | 0.05 | 0.10 | 0.20 | 0.30 | 0.40 |
| (mL) | | | | | |
| Final concentration of | 50.29 | 48.00 | 44.00 | 40.62 | 37.7 |
| DCA-Na (mg/mL) | | | | | |
| Final concentration of | 4.76 | 9.09 | 16.67 | 23.07 | 28.57 |
| L-lysine (mg/mL) | | | | | |
| Group | 1-6 | 1-7 | 1-8 | 1-9 | 1-10 |
| 1% DCA-Na solution | 1.00 | 1.00 | 1.00 | 1.00 | 1.00 |
| (mL) | | | | | |
| 100 mg/mL L-lysine | 0.05 | 0.10 | 0.20 | 0.30 | 0.40 |
| (mL) | | | | | |
| Final concentration of | 10.06 | 9.60 | 8.80 | 8.12 | 7.54 |
| DCA-Na (mg/mL) | | | | | |
| Final concentration of | 4.76 | 9.09 | 16.67 | 23.07 | 28.57 |
| L-lysine (mg/mL) | | | | | |
| Group | 2-1 | 2-2 | 2-3 | 2-4 | 2-5 |
| 5% DCA-Na solution | 1.00 | 1.00 | 1.00 | 1.00 | 1.00 |
| (mL) | | | | | |
| 200 mg/mL L-lysine | 0.05 | 0.10 | 0.20 | 0.30 | 0.40 |
| (mL) | | | | | |
| Final concentration of | 50.29 | 48.00 | 44.00 | 40.62 | 37.7 |
| DCA-Na (mg/mL) | | | | | |
| Final concentration of | 9.52 | 18.18 | 33.33 | 46.15 | 57.14 |
| L-lysine (mg/mL) | | | | | |
| Group | 2-6 | 2-7 | 2-8 | 2-9 | 2-10 |
| 1% DCA-Na solution | 1.00 | 1.00 | 1.00 | 1.00 | 1.00 |
| (mL) | | | | | |
| 200 mg/mL L-lysine | 0.05 | 0.10 | 0.20 | 0.30 | 0.40 |
| (mL) | | | | | |
| Final concentration of | 10.06 | 9.60 | 8.80 | 8.12 | 7.54 |
| DCA-Na (mg/mL) | | | | | |
| Final concentration of | 9.52 | 18.18 | 33.33 | 46.15 | 57.14 |
| L-lysine (mg/mL) | | | | | |
| Group | 3-1 | 3-2 | 3-3 | 3-4 | 3-5 |
| 5% DCA-Na solution | 1.00 | 1.00 | 1.00 | 1.00 | 1.00 |
| (mL) | | | | | |
| 300 mg/mL L-lysine | 0.05 | 0.10 | 0.20 | 0.30 | 0.40 |
| (mL) | | | | | |
| Final concentration of | 50.29 | 48.00 | 44.00 | 40.62 | 37.7 |
| DCA-Na (mg/mL) | | | | | |
| Final concentration of | 14.29 | 27.27 | 50.00 | 69.23 | 85.71 |
| L-lysine (mg/mL) | | | | | |
| Group | 3-6 | 3-7 | 3-8 | 3-9 | 3-10 |
| 1% DCA-Na solution | 1.00 | 1.00 | 1.00 | 1.00 | 1.00 |
| (mL) | | | | | |
| 300 mg/mL L-lysine | 0.05 | 0.10 | 0.20 | 0.30 | 0.40 |
| (mL) | | | | | |
| Final concentration of | 10.06 | 9.60 | 8.80 | 8.12 | 7.54 |
| DCA-Na (mg/mL) | | | | | |
| Final concentration of | 14.29 | 27.27 | 50.00 | 69.23 | 85.71 |
| L-lysine (mg/mL) | | | | | |
| Group | 4-1 | 4-2 | 4-3 | 4-4 | 4-5 |
| 5% DCA-Na solution | 1.00 | 1.00 | 1.00 | 1.00 | 1.00 |
| (mL) | | | | | |
| 400 mg/mL L-lysine | 0.05 | 0.10 | 0.20 | 0.30 | 0.40 |
| (mL) | | | | | |
| Final concentration of | 50.29 | 48.00 | 44.00 | 40.62 | 37.7 |
| DCA-Na (mg/mL) | | | | | |
| Final concentration of | 18.87 | 36.36 | 66.67 | 92.31 | 114.29 |
| L-lysine (mg/mL) | | | | | |
| Group | 4-6 | 4-7 | 4-8 | 4-9 | 4-10 |
| 1% DCA-Na solution | 1.00 | 1.00 | 1.00 | 1.00 | 1.00 |
| (mL) | | | | | |
| 400 mg/mL L-lysine | 0.05 | 0.10 | 0.20 | 0.30 | 0.40 |
| (mL) | | | | | |
| Final concentration of | 10.06 | 9.60 | 8.80 | 8.12 | 7.54 |
| DCA-Na (mg/mL) | | | | | |
| Final concentration of | 18.87 | 36.36 | 66.67 | 92.31 | 114.29 |
| L-lysine (mg/mL) | | | | | |
| Group | 5-1 | 5-2 | 5-3 | 5-4 | 5-5 |
| 5% DCA-Na solution | 1.00 | 1.00 | 1.00 | 1.00 | 1.00 |
| (mL) | | | | | |
| 500 mg/mL L-lysine | 0.05 | 0.10 | 0.20 | 0.30 | 0.40 |
| (mL) | | | | | |
| Final concentration of | 50.29 | 48.00 | 44.00 | 40.62 | 37.7 |
| DCA-Na (mg/mL) | | | | | |
| Final concentration of | 23.81 | 45.45 | 83.33 | 115.38 | 142.86 |
| L-lysine (mg/mL) | | | | | |
| Group | 5-6 | 5-7 | 5-8 | 5-9 | 5-10 |
| 1% DCA-Na solution | 1.00 | 1.00 | 1.00 | 1.00 | 1.00 |
| (mL) | | | | | |
| 500 mg/mL L-lysine | 0.05 | 0.10 | 0.20 | 0.30 | 0.40 |
| (mL) | | | | | |
| Final concentration of | 10.06 | 9.60 | 8.80 | 8.12 | 7.54 |
| DCA-Na (mg/mL) | | | | | |
| Final concentration of | 23.81 | 45.45 | 83.33 | 115.38 | 142.86 |
| L-lysine (mg/mL) | | | | | |

(mg/mL)

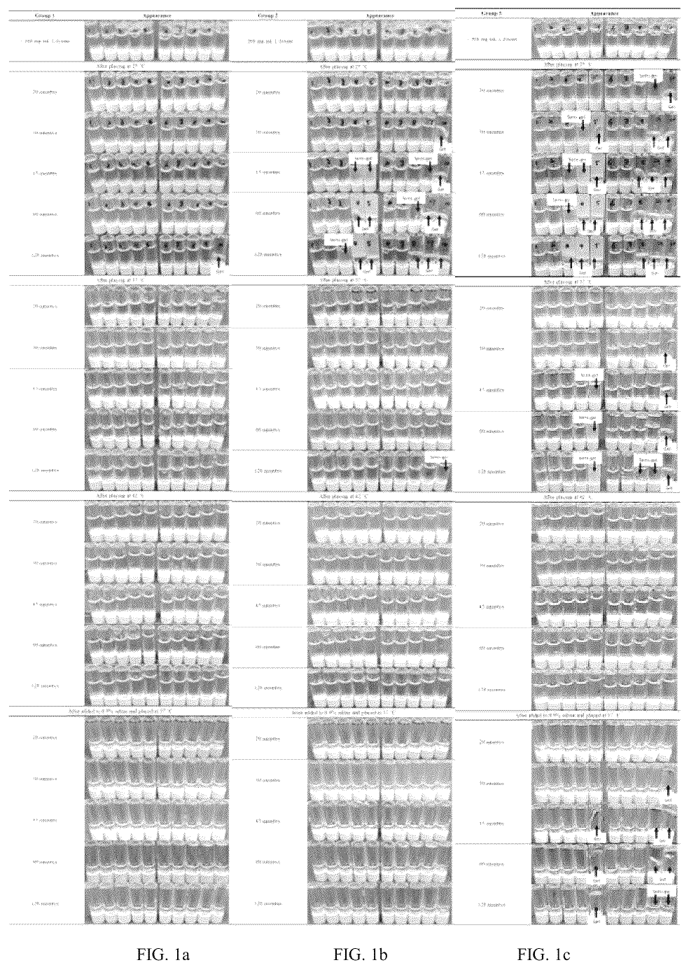

FIG. 1 showed that all groups formed transparent solution when lysine solutions were added to DCA-Na solutions. Mixtures of DCA-Na and lysine with higher concentration of lysine (FIG. 1c-e) started to form gel (remained at the bottom of the bottle after inverted) around 30 minutes when placed at 25° C., while mixtures of DCA-Na and lysine placed at 42° C. did not form gel at all tested lysine concentration in 5% DCA-Na and lysine concentration lower than 140 mg/mL in 1% DCA-Na. Mixtures of 5% DCA-Na and lysine added to 0.9% saline formed gel around 60 minutes at lysine concentration >83 mg/mL and around 30 minutes at lysine concentration >85 mg/mL (FIG. 1c-e). Mixtures of 1% DCA-Na and lysine added to 0.9% saline formed gel around 60 minutes at lysine concentration >45 mg/mL; around 30 minutes at lysine concentration >69 mg/mL and around 20 minutes at lysine concentration >85 mg/mL (FIG. 1c-e). These results showed that higher concentration of LA formed gel in shorter time. Therefore, mixtures of DCA-Na and lysine are suggested to be used as soon as possible after mixing.

In Example 1, the compositions added with 0.9% saline can form gel when the final concentration of DCA-Na was 7.54-44.00 mg/mL, and the final concentration of L-lysine was 45.45-142.86 mg/mL.

To test for the optimal pH values for DCA-Na solutions and L-lysine solutions that form gel after mixing, DCA-Na solutions were mixed with L-lysine with various pH according to TABLE 4.

TABLE 4

| Group | 1 | | | 2 | | | |
|---|---|---|---|---|---|---|---|
| L-lysine (g) | 0.6 | | | 1.2 | | | |
| ddH₂O (mL) | 3.0 | | | 3.0 | | | |
| Final concentration of lysine | 200 | | | 400 | | | |
| (mg/mL) | | | | | | | |
| Initial pH | 10.3-10.4 | | | | | | |
| Group | 1-1 | 1-2 | 1-3 | 1-4 | 1-5 | 1-6 | 1-7 |
| 5% DCA-Na solution (mL) | | | | 1.00 | | | |
| 200 mg/mL L-lysine (mL) | | | | 0.40 | | | |
| Final concentration of | | | | 37.7 | | | |
| DCA-Na (mg/mL) | | | | | | | |
| Final concentration of | | | | 57.14 | | | |
| L-lysine (mg/mL) | | | | | | | |
| pH of L-lysine | 4.0 | 5.0 | 6.0 | 7.0 | 8.0 | 9.0 | 10.0 |
| solution (±0.02) | | | | | | | |
| Group | 1-8 | 1-9 | 1-10 | 1-11 | 1-12 | 1-13 | 1-14 |
| 1% DCA-Na | | | | 1.00 | | | |
| solution (mL) | | | | | | | |
| 200 mg/mL L-lysine (mL) | | | | 0.40 | | | |
| Final concentration | | | | 7.54 | | | |
| of DCA-Na (mg/mL) | | | | | | | |
| Final concentration | | | | 57.14 | | | |
| of L-lysine (mg/mL) | | | | | | | |
| pH of L-lysine | 4.0 | 5.0 | 6.0 | 7.0 | 8.0 | 9.0 | 10.0 |
| solution (±0.02) | | | | | | | |

| Group | 2-1 | 2-2 | 2-3 | 2-4 | 2-5 | 2-6 |
|---|---|---|---|---|---|---|
| 5% DCA-Na | | | 1.00 | | | |
| solution (mL) | | | | | | |
| 200 mg/mL L-lysine (mL) | | | 0.40 | | | |
| Final concentration | | | 37.7 | | | |
| of DCA-Na (mg/mL) | | | | | | |
| Final concentration | | | 114.29 | | | |
| of L-lysine (mg/mL) | | | | | | |
| pH of L-lysine | 5.0 | 6.0 | 7.0 | 8.0 | 9.0 | 10.0 |
| solution (±0.02) | | | | | | |
| Group | 2-7 | 2-8 | 2-9 | 2-10 | 2-11 | 2-12 |
| 1% DCA-Na | | | 1.00 | | | |
| solution (mL) | | | | | | |
| 200 mg/mL L-lysine (mL) | | | 0.40 | | | |
| Final concentration | | | 7.54 | | | |
| of DCA-Na (mg/mL) | | | | | | |
| Final concentration | | | 114.29 | | | |
| of L-lysine (mg/mL) | | | | | | |
| pH of L-lysine | 5.0 | 6.0 | 7.0 | 8.0 | 9.0 | 10.0 |
| solution (±0.02) | | | | | | |

* DCA-Na solutions mixed with pH 3.0 L-lysine solution (200 mg/mL) formed precipitation.
* DCA-Na solutions mixed with pH 4.0 L-lysine solution (400 mg/mL) formed precipitation.

TABLE 5

| | pH of L-lysine solution | | | | | | |
|---|---|---|---|---|---|---|---|
| | 4.0 | 5.0 | 6.0 | 7.0 | 8,0 | 9.0 | 10.0 |
| Group | 1-1 | 1-2 | 1-3 | 1-4 | 1-5 | 1-6 | 1-7 |
| pH of L-lysine | 7.21 | 7.35 | 7.38 | 7.58 | 8.09 | 9.05 | 9.97 |
| solution + 5% | | | | | | | |
| DCA-Na solution | | | | | | | |
| Group | 1-8 | 1-9 | 1-10 | 1-11 | 1-12 | 1-13 | 1-14 |
| pH of L-lysine | 6.71 | 7.02 | 7.09 | 7.42 | 8.05 | 8.98 | 9.92 |
| solution + 1% | | | | | | | |
| DCA-Na solution | | | | | | | |

| | pH of L-lysine solution | | | | | |
|---|---|---|---|---|---|---|
| | 5.0 | 6.0 | 7.0 | 8.0 | 9.0 | 10.0 |
| Group | 2-1 | 2-2 | 2-3 | 2-4 | 2-5 | 2-6 |
| pH of L-lysine | 7.45 | 7.55 | 7.70 | 8.26 | 9.07 | 9.92 |
| solution + 5% | | | | | | |
| DCA-Na solution | | | | | | |
| Group | 2-7 | 2-8 | 2-9 | 2-10 | 2-11 | 2-12 |
| pH of L-lysine | 6.96 | 7.22 | 7.50 | 8.23 | 9.04 | 9.89 |
| solution + 1% | | | | | | |
| DCA-Na solution | | | | | | |

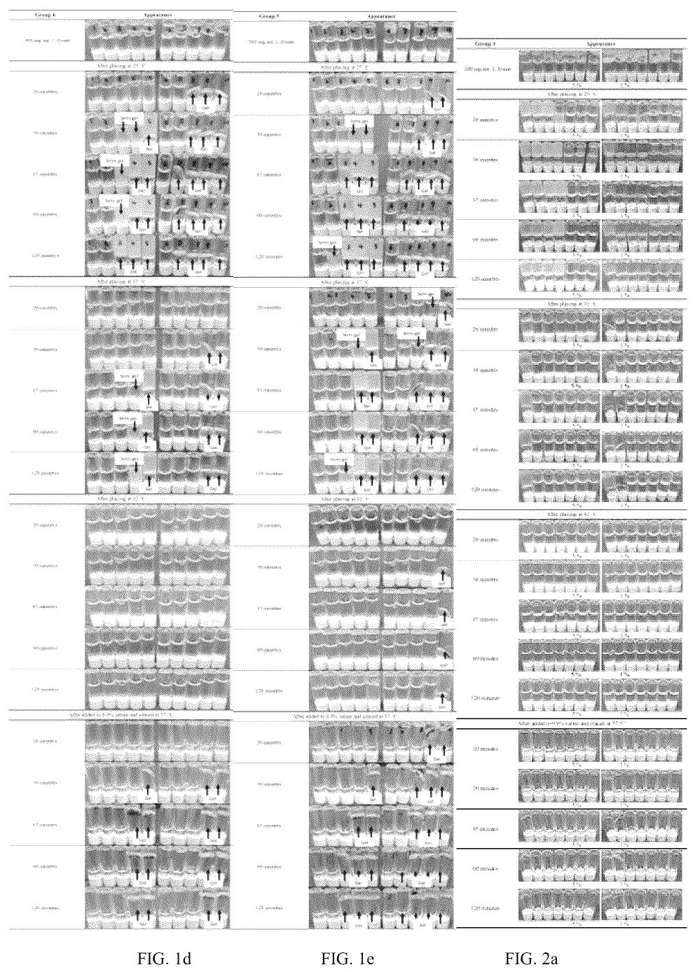

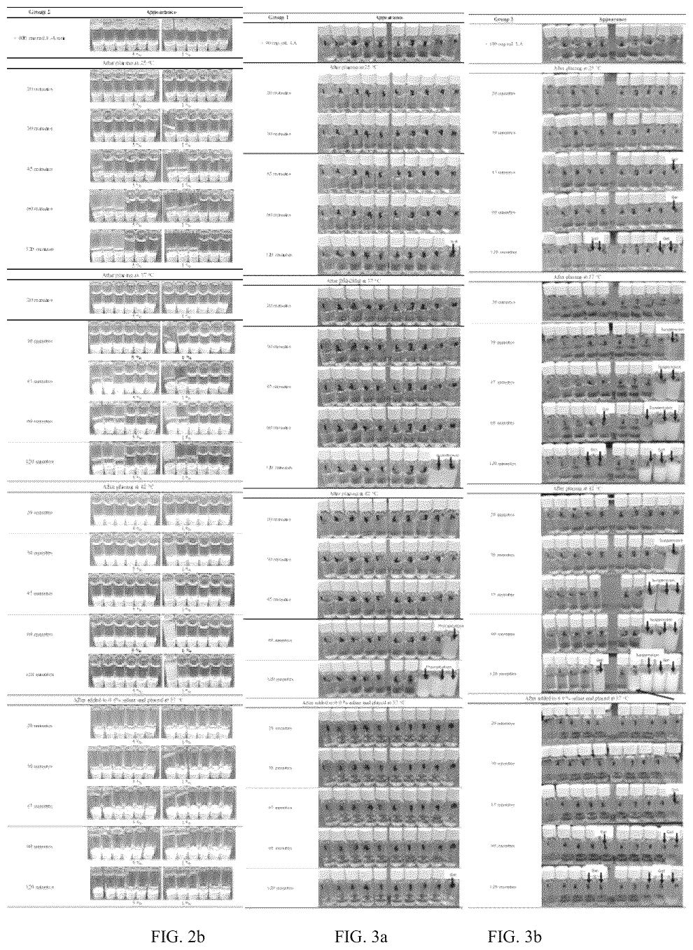

FIG. 2 showed that all groups formed transparent solution when lysine solutions were added to DCA-Na solutions. The pH value of mixed solutions ranged from 7.21-9.97 and 6.71-9.92 in 5% and 1% DCA-Na solution mixed with 200 mg/mL L-lysine solution at pH ranged from 4.0 to 10.0; 7.45-9.92 and 6.96-9.89 in 5% and 1% DCA-Na solution mixed with 400 mg/mL L-lysine solution at pH ranged from 5.0 to 10.0 (TABLE 5). At 200 mg/mL L-lysine test, 5% DCA-Na and L-lysine added to 0.9% saline formed gel around 60 minutes at pH 4.0; mixtures of 1% DCA-Na and L-lysine added to 0.9% saline formed gel around 30 minutes at pH 4.0, around 45 minutes at pH 5.0 (FIG. 2a). At 400 mg/mL L-lysine test, mixtures of 5% DCA-Na and L-lysine added to 0.9% saline formed gel around 45 minutes at pH 5.0 and 6.0, around 60 minutes at pH 7.0; mixtures of 1% DCA-Na and L-lysine added to 0.9% saline formed gel around 30 minutes at pH 5.0, around 45 minutes at pH 6.0 (FIG. 2b). Therefore, suitable pH for L-lysine solution before mixing is <8.0, preferably 5.0-7.0, more preferably around pH 5.0-6.0. Lower pH value is suggested for lower concentration of L-lysine.

The compositions added with 0.9% saline can form gel when the final pH of the composition was 7.02-7.70.

### Example 2. Compositions of DCA-Na and Lysine Aspirin

To test if mixing DCA-Na solutions with lysine-containing NSAID can form gel, DCA-Na solutions were mixed with LA (Lyacety, 0.9 g/bottle, equivalent to 0.5 g aspirin, China Chemical & Pharmaceutical Co., Ltd., Taipei City, China) according to TABLE 6.

TABLE 6

| Group | 1 | 2 | 3 | 4 | 5 |
|---|---|---|---|---|---|
| Lysine aspirin (g) | 0.9 | 0.9 | 0.9 | 0.9 | 0.9 |
| ddH2O (mL) | 10.0 | 5.0 | 3.0 | 2.0 | 1.5 |
| Final concentration of | 90 | 180 | 300 | 450 | 600 |
| LA (mg/mL) | | | | | |
| Group | 1-1 | 1-2 | 1-3 | 1-4 | 1-5 |
| 5% DCA-Na solution | 1.00 | 1.00 | 1.00 | 1.00 | 1.00 |
| (mL) | | | | | |
| 90 mg/mL LA (mL) | 0.05 | 0.10 | 0.20 | 0.30 | 0.40 |
| Final concentration of | 50,29 | 48.00 | 44.00 | 40.62 | 37.7 |
| DCA-Na (mg/mL) | | | | | |
| Final concentration of | 4.29 | 8.18 | 15.00 | 20.77 | 25.71 |
| LA (mg/mL) | | | | | |
| Group | 1-6 | 1-7 | 1-8 | 1-9 | 1-10 |
| 1% DCA-Na solution | 1.00 | 1.00 | 1.00 | 1.00 | 1.00 |
| (mL) | | | | | |
| 90 mg/mL LA (mL) | 0.05 | 0.10 | 0.20 | 0.30 | 0.40 |
| Final concentration of | 10.06 | 9.60 | 8.80 | 8.12 | 7.54 |
| DCA-Na (mg/mL) | | | | | |
| Final concentration of | 4.29 | 8.18 | 15.00 | 20.77 | 25.71 |
| LA (mg/mL) | | | | | |
| Group | 2-1 | 2-2 | 2-3 | 2-4 | 2-5 |
| 5% DCA-Na solution | 1.00 | 1.00 | 1.00 | 1.00 | 1.00 |
| (mL) | | | | | |
| 180 mg/mL LA (mL) | 0.05 | 0.10 | 0.20 | 0.30 | 0.40 |
| Final concentration of | 50.29 | 48.00 | 44.00 | 40.62 | 37.7 |
| DCA-Na (mg/mL) | | | | | |
| Final concentration of | 8.57 | 16.36 | 30.00 | 41.54 | 51.43 |
| LA (mg/mL) | | | | | |
| Group | 2-6 | 2-7 | 2-8 | 2-9 | 2-10 |
| 1% DCA-Na solution | 1.00 | 1.00 | 1.00 | 1.00 | 1.00 |
| (mL) | | | | | |
| 180 mg/mL LA (mL) | 0.05 | 0.10 | 0.20 | 0.30 | 0.40 |
| Final concentration of | 10.06 | 9.60 | 8.80 | 8.12 | 7.54 |
| DCA-Na (mg/mL) | | | | | |
| Final concentration of | 8.57 | 16.36 | 30.00 | 41.54 | 51.43 |
| LA (mg/mL) | | | | | |
| Group | 3-1 | 3-2 | 3-3 | 3-4 | 3-5 |
| 5% DCA-Na solution | 1.00 | 1.00 | 1.00 | 1.00 | 1.00 |
| (mL) | | | | | |
| 300 mg/mL LA (mL) | 0.05 | 0.10 | 0.20 | 0.30 | 0.40 |
| Final concentration of | 50.29 | 48.00 | 44.00 | 40.62 | 37.7 |
| DCA-Na (mg/mL) | | | | | |
| Final concentration of | 14.29 | 27.27 | 50.00 | 69.23 | 85.71 |
| LA (mg/mL) | | | | | |
| Group | 3-6 | 3-7 | 3-8 | 3-9 | 3-10 |
| 1% DCA-Na solution | 1.00 | 1.00 | 1.00 | 1.00 | 1.00 |
| (mL) | | | | | |
| 300 mg/mL LA (mL) | | | | | |
| Final concentration of | 10.06 | 9.60 | 8.80 | 8.12 | 7.54 |
| DCA-Na (mg/mL) | | | | | |
| Final concentration of | 14.29 | 27.27 | 50.00 | 69.23 | 85.71 |
| LA (mg/mL) | | | | | |
| Group | 4-1 | 4-2 | 4-3 | 4-4 | 4-5 |
| 5% DCA-Na solution | 1.00 | 1.00 | 1.00 | 1.00 | 1.00 |
| (mL) | | | | | |
| 450 mg/mL LA (mL) | 0.05 | 0.10 | 0.20 | 0.30 | 0.40 |
| Final concentration of | 50.29 | 48.00 | 44.00 | 40.62 | 37.7 |
| DCA-Na(mg/mL) | | | | | |
| Final concentration of | 21.43 | 40.91 | 75.00 | 103.85 | 128.57 |
| LA (mg/mL) | | | | | |
| Group | 4-6 | 4-7 | 4-8 | 4-9 | 4-10 |
| 1% DCA-Na solution | 1.00 | 1.00 | 1.00 | 1.00 | 1.00 |
| (mL) | | | | | |
| 450 mg/mL LA (mL) | 0.05 | 0.10 | 0.20 | 0.30 | 0.40 |
| Final concentration of | 10.06 | 9.60 | 8.80 | 8.12 | 7.54 |
| DCA-Na (mg/mL) | | | | | |
| Final concentration of | 21.43 | 40.91 | 75.00 | 103.85 | 128.57 |
| LA (mg/mL) | | | | | |
| Group | 5-1 | 5-2 | 5-3 | 5-4 | 5-5 |
| 5% DCA-Na solution | 1.00 | 1.00 | 1.00 | 1.00 | 1.00 |
| (mL) | | | | | |
| 600 mg/mL LA (mL) | 0.05 | 0.10 | 0.20 | 0.30 | 0.40 |
| Final concentration of | 50.29 | 48.00 | 44.00 | 40.62 | 37.7 |
| DCA-Na (mg/mL) | | | | | |
| Final concentration of | 28.57 | 54.55 | 100.00 | 138.46 | 171.43 |
| LA (mg/mL) | | | | | |
| Group | 5-6 | 5-7 | 5-8 | 5-9 | 5-10 |
| 1% DCA-Na solution | 1.00 | 1.00 | 1.00 | 1.00 | 1.00 |
| (mL) | | | | | |
| 600 mg/mL LA (mL) | 0.05 | 0.10 | 0.20 | 0.30 | 0.40 |
| Final concentration of | 10.06 | 9.60 | 8.80 | 8.12 | 7.54 |
| DCA-Na (mg/mL) | | | | | |
| Final concentration of | 28.57 | 54.55 | 100.00 | 138.46 | 171.43 |
| LA (mg/mL) | | | | | |

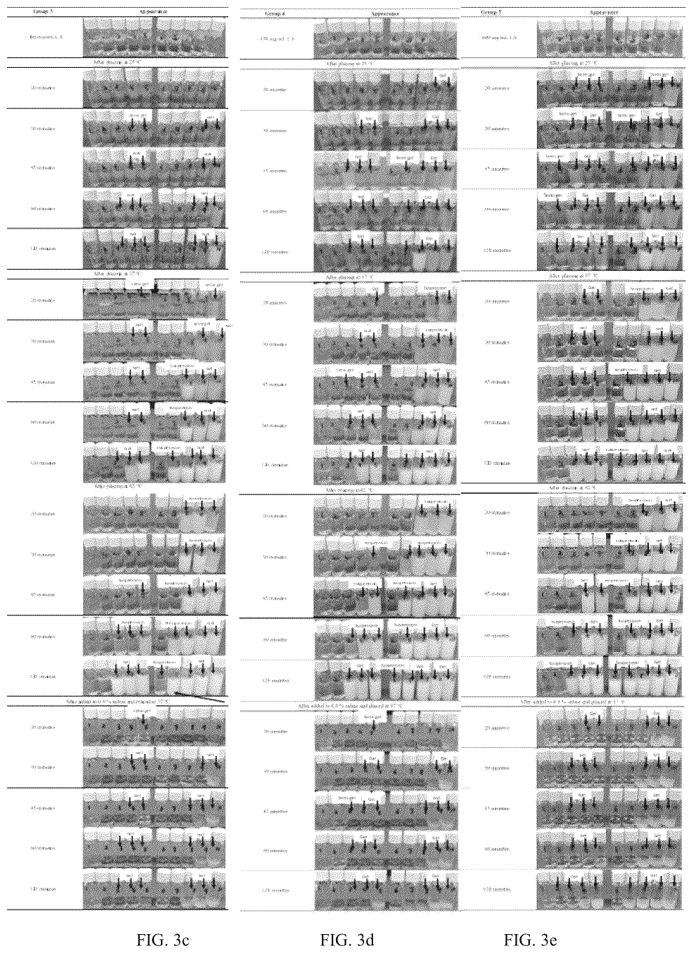

FIG. 3 showed that all groups formed transparent solution when LA solutions were added to DCA-Na solutions. Mixtures of DCA-Na and LA with higher concentration of LA started to form gel around 20 minutes when placed at 25° C. (FIG. 3d, e), while mixtures placed at 37 or 42° C. took longer time to form gel but formed suspension (or precipitation) within a short period of time (FIG. 3b-e). Mixtures added to 0.9% saline formed gel around 60 minutes at LA concentration ≥50 mg/mL; around 30 minutes at LA concentration >69 mg/mL (FIG. 3b-e). Higher concentration of LA formed gel in shorter time. Therefore, mixtures of DCA-Na and LA are suggested to be used as soon as possible after mixing.

In Example 2, the compositions added with 0.9% saline can form gel when the final concentration of DCA-Na was 7.54-48.00 mg/mL, even up to 50.29 mg/mL; and the final concentration of LA was 25.71-171.43 mg/mL, wherein the final concentrations of lysine and aspirin were about 11.40-76.81 mg/mL and 14.31-94.62 mg/mL, respectively.

### Example 3. Compositions of DCA-Na and Lysine Aspirin with Lidocaine HCl

To test if DCA-Na solutions and LA dissolved in local anesthetic lidocaine HCl form gel after mixing, DCA-Na solutions were mixed with LA in lidocaine HCl (5 mL/bottle, Lita Pharmacy CO., Ltd., Taichung City, China) according to TABLE 7.

TABLE 7

| Group | 1 | 2 | 3 | 4 | 5 |
|---|---|---|---|---|---|
| Lysine aspirin (g) | 0.9 | 0.9 | 0.9 | 0.9 | 0.9 |
| Lidocaine HCL(mL) | 10.0 | 5.0 | 3.0 | 2.0 | 1.5 |
| Final concentration | 90 | 180 | 300 | 450 | 600 |
| of LA (mg/mL) | | | | | |
| Group | 1-1 | 1-2 | 1-3 | 1-4 | 1-5 |
| 5% DCA-Na solution | 1.000 | 1.000 | 1.000 | 1.000 | 1.000 |
| (mL) | | | | | |
| 90 mg/mL LA/lidocaine | 0.176 | 0.200 | 0.300 | 0.400 | 0.428 |
| (mL) | | | | | |
| Final concentration | 44.90 | 44.00 | 40.6 | 37.71 | 36.97 |
| of DCA-Na (mg/mL) | | | | | |
| Final concentration | 13.47 | 15.00 | 20.77 | 25.71 | 26.97 |
| of LA (mg/mL) | | | | | |
| Final concentration | 2.99 | 3.33 | 4.62 | 5.72 | 5.99 |
| of lidocaine (mg/mL) | | | | | |
| Group | 1-6 | 1-7 | 1-8 | 1-9 | 1-10 |
| 1% DCA-Na solution | 1.000 | 1.000 | 1.000 | 1.000 | 1.000 |
| (mL) | | | | | |
| 90 mg/mL LA/lidocaine | 0.176 | 0.200 | 0.300 | 0.400 | 0.428 |
| (mL) | | | | | |
| Final concentration | 8.98 | 8.80 | 8.12 | 7.54 | 7.39 |
| of DCA-Na (mg/mL) | | | | | |
| Final concentration | 13.47 | 15.00 | 20.77 | 25.71 | 26.97 |
| of LA (mg/mL) | | | | | |
| Final concentration | 2.99 | 3.33 | 4.62 | 5.72 | 5.99 |
| of lidocaine (mg/mL) | | | | | |
| Group | 2-1 | 2-2 | 2-3 | 2-4 | 2-5 |
| 5% DCA-Na solution | 1.000 | 1.000 | 1.000 | 1.000 | 1.000 |
| (mL) | | | | | |
| 180 mg/mL LA/lidocaine | 0.176 | 0.200 | 0.300 | 0.400 | 0.428 |
| (mL) | | | | | |
| Final concentration | 44.90 | 44.00 | 40.6 | 37.71 | 36.97 |
| of DCA-Na (mg/mL) | | | | | |
| Final concentration | 26.94 | 30.00 | 41.54 | 51.43 | 53.95 |
| of LA (mg/mL) | | | | | |
| Final concentration | 2.99 | 3.33 | 4.62 | 5.72 | 5.99 |
| of lidocaine (mg/mL) | | | | | |
| Group | 2-6 | 2-7 | 2-8 | 2-9 | 2-10 |
| 1% DCA-Na solution | 1.000 | 1.000 | 1.000 | 1.000 | 1.000 |
| (mL) | | | | | |
| 180 mg/mL LA/lidocaine | 0.176 | 0.200 | 0.300 | 0.400 | 0.428 |
| (mL) | | | | | |
| Final concentration | 8.98 | 8.80 | 8.12 | 7.54 | 7.39 |
| of DCA-Na (mg/mL) | | | | | |
| Final concentration | 26.94 | 30.00 | 41.54 | 51.43 | 53.95 |
| of LA (mg/mL) | | | | | |
| Final concentration | 2.99 | 3.33 | 4.62 | 5.72 | 5.99 |
| of lidocaine (mg/mL) | | | | | |
| Group | 3-1 | 3-2 | 3-3 | 3-4 | 3-5 |
| 5% DCA-Na solution | 1.000 | 1.000 | 1.000 | 1.000 | 1.000 |
| (mL) | | | | | |
| 300 mg/mL LA/lidocaine | 0.176 | 0.200 | 0.300 | 0.400 | 0.428 |
| (mL) | | | | | |
| Final concentration | 44.90 | 44.00 | 40.6 | 37.71 | 36.97 |
| of DCA-Na (mg/mL) | | | | | |
| Final concentration | 44.90 | 50.00 | 69.23 | 85.71 | 89.92 |
| of LA (mg/mL) | | | | | |
| Final concentration | 2.99 | 3.33 | 4.62 | 5.72 | 5.99 |
| of lidocaine (mg/mL) | | | | | |
| Group | 3-6 | 3-7 | 3-8 | 3-9 | 3-10 |
| 1% DCA-Na solution | 1.000 | 1.000 | 1.000 | 1.000 | 1.000 |
| (mL) | | | | | |
| 300 mg/mL LA/lidocaine | 0.176 | 0.200 | 0.300 | 0.400 | 0.428 |
| (mL) | | | | | |
| Final concentration | 8.98 | 8.80 | 8.12 | 7.54 | 7.39 |
| of DCA-Na (mg/mL) | | | | | |
| Final concentration | 44.90 | 50.00 | 69.23 | 85.71 | 89.92 |
| of LA (mg/mL) | | | | | |
| Final concentration | 2.99 | 3.33 | 4.62 | 5.72 | 5.99 |
| of lidocaine (mg/mL) | | | | | |
| Group | 4-1 | 4-2 | 4-3 | 4-4 | 4-5 |
| 5% DCA-Na solution | 1.000 | 1.000 | 1.000 | 1.000 | 1.000 |
| (mL) | | | | | |
| 450 mg/mL LA/lidocaine | 0.176 | 0.200 | 0.300 | 0.400 | 0.428 |
| (mL) | | | | | |
| Final concentration | 44.90 | 44.00 | 40.6 | 37.71 | 36.97 |
| of DCA-Na (mg/mL) | | | | | |
| Final concentration | 67.35 | 75.00 | 103.85 | 128.57 | 134.87 |
| of LA (mg/mL) | | | | | |
| Final concentration | 2.99 | 3.33 | 4.62 | 5.72 | 5.99 |
| of lidocaine (mg/mL) | | | | | |
| Group | 4-6 | 4-7 | 4-8 | 4-9 | 4-10 |
| 1% DCA-Na solution | 1.000 | 1.000 | 1.000 | 1.000 | 1.000 |
| (mL) | | | | | |
| 450 mg/mL LA/lidocaine | 0.176 | 0.200 | 0.300 | 0.400 | 0.428 |
| (mL) | | | | | |
| Final concentration | 8.98 | 8.80 | 8.12 | 7.54 | 7.39 |
| of DCA-Na (mg/mL) | | | | | |
| Final concentration | 67.35 | 75.00 | 103.85 | 128.57 | 134.87 |
| of LA (mg/mL) | | | | | |
| Final concentration | 2.99 | 3.33 | 4.62 | 5.72 | 5.99 |
| of lidocaine (mg/mL) | | | | | |
| Group | 5-1 | 5-2 | 5-3 | 5-4 | 5-5 |
| 5% DCA-Na solution | 1.000 | 1.000 | 1.000 | 1.000 | 1.000 |
| (mL) | | | | | |
| 600 mg/mL LA/lidocaine | 0.176 | 0.200 | 0.300 | 0.400 | 0.428 |
| (mL) | | | | | |
| Final concentration | 44.90 | 44.00 | 40.6 | 37.71 | 36.97 |
| of DCA-Na (mg/mL) | | | | | |
| Final concentration | 89.80 | 100.00 | 138.46 | 171.43 | 179.83 |
| of LA (mg/mL) | | | | | |
| Final concentration | 2.99 | 3.33 | 4.62 | 5.72 | 5.99 |
| of lidocaine (mg/mL) | | | | | |
| Group | 5-6 | 5-7 | 5-8 | 5-9 | 5-10 |
| 1% DCA-Na solution | 1.000 | 1.000 | 1.000 | 1.000 | 1.000 |
| (mL) | | | | | |
| 600 mg/mL LA/lidocaine | 0.176 | 0.200 | 0.300 | 0.400 | 0.428 |
| (mL) | | | | | |
| Final concentration | 8.98 | 8.80 | 8.12 | 7.54 | 7.39 |
| of DCA-Na (mg/mL) | | | | | |
| Final concentration | 89.80 | 100.00 | 138.46 | 171.43 | 179.83 |
| of LA (mg/mL) | | | | | |
| Final concentration | 2.99 | 3.33 | 4.62 | 5.72 | 5.99 |
| of lidocaine (mg/mL) | | | | | |

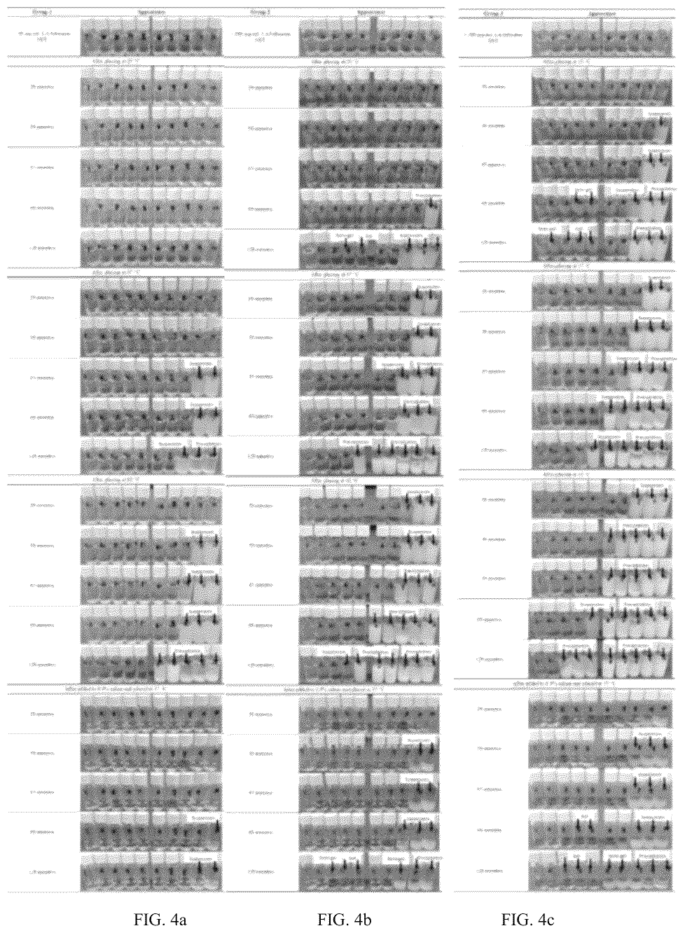

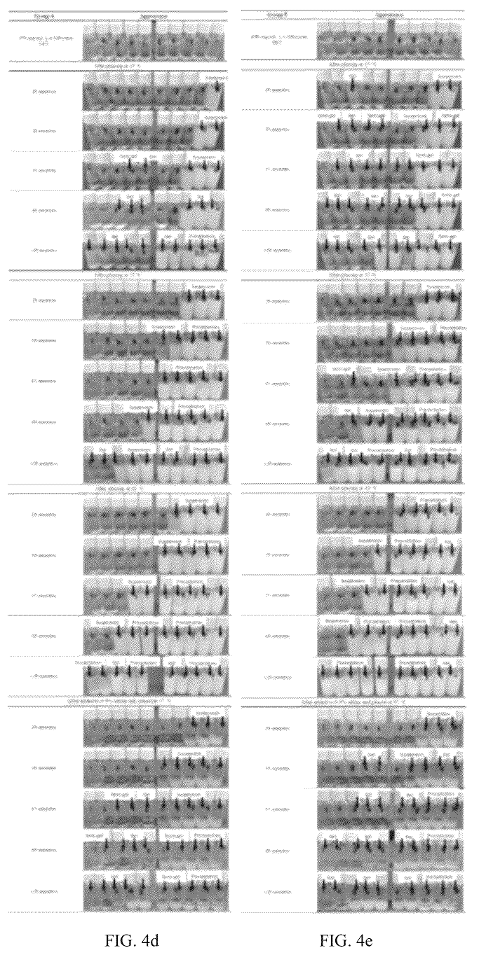

FIG. 4 showed that all groups formed transparent solution when LA in lidocaine HCl solutions were added to DCA-Na solutions. Mixtures of DCA-Na and LA in lidocaine HCl with high concentration of LA started to form gel around 30 minutes when placed at 25° C. (FIG. 4e), while mixtures placed at 37 or 42° C. took longer time to form gel but formed suspension or precipitation within a short period of time (FIG. 4a-e). For 5% DCA-Na solution, mixtures of DCA-Na and LA in lidocaine HCl added to 0.9% saline formed gel around 60 minutes at LA concentration >70 mg/mL; around 45 minutes at LA concentration >134 mg/mL; around 30 minutes at LA concentration >170 mg/mL. (FIG. 4c-e). Concentration of lidocaine HCl mixing with 5% DCA-Na solution was tolerable up to 6 mg/mL. For 1% DCA-Na solution, mixtures of DCA-Na and LA+lidocaine HCl added to 0.9% saline formed gel around 120 minutes at LA concentration >40 mg/mL; around 60 minutes at LA concentration >67 mg/mL; around 45 minutes at LA concentration >85 mg/mL (FIG. 4b-e). Suitable concentration of lidocaine HCl mixing with 1% DCA-Na solution was around 3 mg/mL. These results showed that high concentration of lidocaine added to low concentration of DCA-Na precipitated easily.

In Example 3, the compositions added with 0.9% saline can form gel when the final concentration of DCA-Na was 8.12-44.90 mg/mL; the final concentration of LA was 41.54-179.83 mg/mL, wherein the final concentrations of lysine and aspirin were about 18.61-80.61 mg/mL and 22.93-99.22 mg/mL, respectively; and the final concentration of lidocaine was 2.99-6.99 mg/mL.

### Example 4. Effects of DCA-Na and Lysine Aspirin with Lidocaine HCl in Porcine Tissue

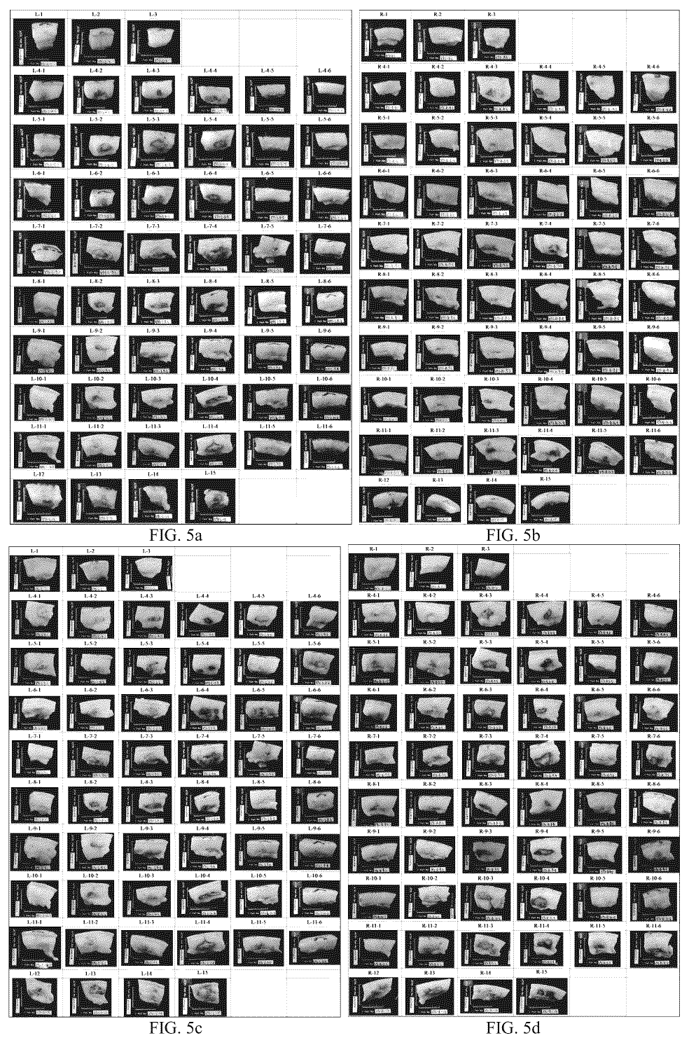

2 male SPF Landrace pigs aging around 5-6 months were anesthetized via intramuscular injection of 0.04 mg/kg Atropine. After 10-15 minutes, 6 mg/kg Zoletil 50 and 2.2 mg/kg Rompun were injected intramuscularly. 1.5 mL lidocaine HCl were added to LA and mixed until dissolved. 0.35 mL lidocaine HCl/LA solution were added to 2 mL 1% or 5% DCA-Na solution and mixed until dissolved. Pigs were injected with 0.9% saline, 1% or 5% DCA-Na solutions with or without lidocaine HCl/LA at different time points according to TABLE 8. Area for each injection site is 16 cm2 and compositions were injected at a depth of 1.0 cm at the center of each site. 55 sites were injected at each side of the pigs (Total 110 sites/pig). After sacrificed (day 0), fat tissue samples were collected and cut in half from the center. Photos of sections were recorded and shown in FIG. 5.

TABLE 8

| | | | | | | | | Amount |
|---|---|---|---|---|---|---|---|---|
| | | | | | | | | of |
| Group | Composition | Time points for injection | | | | | | single |
| 1 | Blank | — | | | | | | — |
| 2 | 0.9% Saline | −3 hour | | | | | | 200 |
| 3 | 0.9% Saline | −3 hour | | | | | | 400 |
| 4-1~4-6 | 1% DCA-Na | 4-1 | 4-2 | 4-3 | 4-4 | 4.5 | 4-6 | 200 |
| | solution | −28 day | −21 day | −14 day | −7 day | −2 day | −3 hour | |
| 5-1~5-6 | 1% DCA-Na | 5-1 | 5-2 | 5-3 | 5-4 | 5-5 | 5-6 | 400 |
| | solution | −28 day | −21 day | −14 day | −7 day | −2 day | −3 hour | |
| 6-1~6-6 | 5% DCA-Na | 6-1 | 6-2 | 6-3 | 6-4 | 6-5 | 6-6 | 200 |
| | solution | −28 day | −21 day | −14 day | −7 day | −2 day | 3 hour | |
| 7-1~7-6 | 65% DCA-Na | 7-1 | 7-2 | 7-3 | 7-4 | 7-5 | 7-6 | 400 |
| | solution | −28 day | −21 day | −14 day | −7 day | −2 day | −3 hour | |
| 8-1~8-6 | 1% DCA-Na | 8-1 | 8-2 | 8-3 | 8-4 | 8~5 | 8~6 | 200 |
| | solution + LA in | | | | | | | |
| | Lidocaine HCl | −28 day | 21 day | −14 day | −7 day | −2 day | 3 hour | |
| 9-1~9-6 | 1% DCA-Na | 9-1 | 9-2 | 9-3 | 9-4 | 9-5 | 9-6 | 400 |
| | solution + LA in | | | | | | | |
| | Lidocaine HCl | −28 day | 21 day | −14 day | −7 day | −2 day | −3 hour | |
| 10-1~10-6 | 5% DCA-Na | 10-1 | 10-2 | 10-3 | 10-4 | 10-5 | 10-6 | 200 |
| | solution + LA in | | | | | | | |
| | Lidocaine HCl | −28 day | −21 day | −14 day | −7 day | −2 day | −3 hour | |
| 11-1~11-6 | 5% DCA-Na | 11-1 | 11-2 | 11-3 | 11-4 | 11-5 | 11-6 | 400 |
| | solution + LA in | | | | | | | |
| | Lidocaine HCl | −28 day | −21 day | −14 day | −7 day | −2 day | −3 hour | |

| | | | |
|---|---|---|---|
| 12 | 1% DCA-Na | −28 day, −21 day, −14 day , −7 day | 200 |
| | solution + LA in | | |
| | Lidocaine HCl | | |
| 13 | 1% DCA-Na | −28 day, −21 day, −14 day , −7 day | 400 |
| | solution + LA in | | |
| | Lidocaine HCl | | |
| 14 | 5% DCA-Na | −28 day, −21 day, −14 day , −7 day | 200 |
| | solution + LA in | | |
| | Lidocaine HCl | | |
| 15 | 5% DCA-Na | −28 day, −21 day, −14 day , −7 day | 400 |
| | solution + LA in | | |

Lidocaine HCl

As shown in FIG. 5, 7 days after injection, sites injected with DCA-Na solution alone were slightly harder and much swollen than sites injected with DCA-Na solutions with lidocaine HCl/LA. FIG. 5 showed that cytolysis occurs at injected site if DCA-Na solutions were injected alone (Group 4-7). On the other hand, cytolysis occurs at the bottom of fat tissue if DCA-Na solutions were injected along with lidocaine HCl/LA (Group 8-15). This might suggest that DCA-Na solutions alone tend to diffuse in fat tissue, however, mixing DCA-Na solutions with lidocaine HCl/LA formed gel that might deposit and diffuse at the bottom of the fat tissue, which coincide with less hardness being palpated. Cytolysis and/or inflammation at sites injected with DCA-Na solution alone were observable for at least 21-28 days but were less observable after 21 days at sites injected with DCA-Na solutions with lidocaine HCl/LA.

Compositions of DCA-Na and Lysine Aspirin with Lidocaine HCl can effectively reduce fat, with less adverse effects, such as inflammation.

### Example 5. Compositions of DCA-Na and L-lysine with DSP

To test if mixing DCA-Na solutions with lysine and another anti-inflammatory drug, DSP (Tai Yu Chemical & Pharmaceutical Co., Ltd., Hsinchu County, China), can form gel and its optimal pH value for forming gel, DCA-Na solutions were mixed with L-lysine/DSP of different pH values according to TABLE 9. Requirement: Final concentration of DSP: ≤1 mg/mL.

TABLE 9

| Group | 1 | 2 |
|---|---|---|
| L-lysine (g) | 0.6 | 1.2 |
| DSP (mg) | 13.0 | 13.0 |
| ddH₂O (mL) | 3.0 | 3.0 |
| Final concentration of L-lysine | 200 | 400 |
| (mg/mL) | | |
| Final concentration of DSP | 4.33 | 4.33 |
| (mg/mL) | | |

| | | | | |
|---|---|---|---|---|
| Group | 1-1 | 1-2 | 1-3 | 1-4 |

| | |
|---|---|
| 5% DCA-Na solution (mL) | 1.00 |
| L-lysine (200 mg/mL)/DSP solution | 0.30 |

(mL)

| | |
|---|---|
| Final concentration of DCA-Na | 40.615 |

(mg/mL)

| | |
|---|---|
| Final concentration of L-lysine | 46.154 |

(mg/mL)

| | |
|---|---|
| Final concentration of DSP (mg/mL) | 0.999 |

| | | | | |
|---|---|---|---|---|
| pH of L-lysine solution (+ 0.10) | 4.0 | 5.0 | 6.0 | 7.0 |

| | | | | |
|---|---|---|---|---|
| Group | 1-5 | 1-6 | 1-7 | 1-8 |

| | |
|---|---|
| 1% DCA-Na solution (mL) | 1.00 |
| L-lysine (200 mg/mL)/DSP solution | 0.30 |

(mL)

| | |
|---|---|
| Final concentration of DCA-Na | 8.123 |

(mg/mL)

| | |
|---|---|
| Final concentration of L-lysine | 46.154 |

(mg/mL)

| | |
|---|---|
| Final concentration of DSP (mg/mL) | 0.999 |

| pH of L-lysine solution (±0.02) | 4.0 | 5.0 | 6.0 | 7.0 |
|---|---|---|---|---|
| Group | 2-1 | 2-2 | 2-3 | 2-4 |

| | |
|---|---|
| 5% DCA-Na solution (mL) | 1.00 |
| L-lysine (400 mg/mL)/DSP solution | 0.30 |

(mL)

| | |
|---|---|
| Final concentration of DCA-Na | 40.615 |

(mg/mL)

| | |
|---|---|
| Final concentration of L-lysine | 92.308 |

| (mg/mL) | | | | |
|---|---|---|---|---|
| Final concentration of DSP (mg/mL) | 4.0 | 5.0 | 6.0 | 7.0 |
| Group | 2-5 | 2-6 | 2-7 | 2-8 |

| | |
|---|---|
| 1% DCA-Na solution (mL) | 1.00 |
| L-lysine (200 mg/mL)/DSP solution | 0.30 |

(mL)

| | |
|---|---|
| Final concentration of DCA-Na | 8.123 |

(mg/mL)

| | |
|---|---|
| Final concentration of L-lysine | 92.308 |

(mg/mL)

| | |
|---|---|
| Final concentration of DSP (mg/mL) | 0.999 |

| | | | | |
|---|---|---|---|---|
| pH of L-lysine solution (±0.02) | 4.0 | 5.0 | 6.0 | 7.0 |

TABLE 10

| pH of L-lysine solution | 4.0 | 5.0 | 6.0 | 7.0 |
|---|---|---|---|---|
| Group | 1-1 | 1-2 | 1-3 | 1-4 |
| pH of L-lysine/DSP | 6.87 | 7.15 | 7.21 | 7.43 |
| solution + 5% DCA-Na | | | | |
| solution | | | | |
| Group | 1-5 | 1-6 | 1-7 | 1-8 |
| pH of L-lysine/DSP | 6.48 | 6.81 | 6.96 | 7.28 |
| solution + 1% DCA-Na | | | | |
| solution | | | | |
| pH of L-lysine solution | 4.0 | 5.0 | 6.0 | 7.0 |
| Group | 2-1 | 2-2 | 2-3 | 2-4 |
| pH of L-lysine/DSP | 7.11 | 7.27 | 7.38 | 7.54 |
| solution + 5% DCA-Na | | | | |
| solution | | | | |
| Group | 2-5 | 2-6 | 2-7 | 2-8 |
| pH of L-lysine/DSP | 6.75 | 7.01 | 7.17 | 7.34 |
| solution + 1% DCA-Na | | | | |
| solution | | | | |

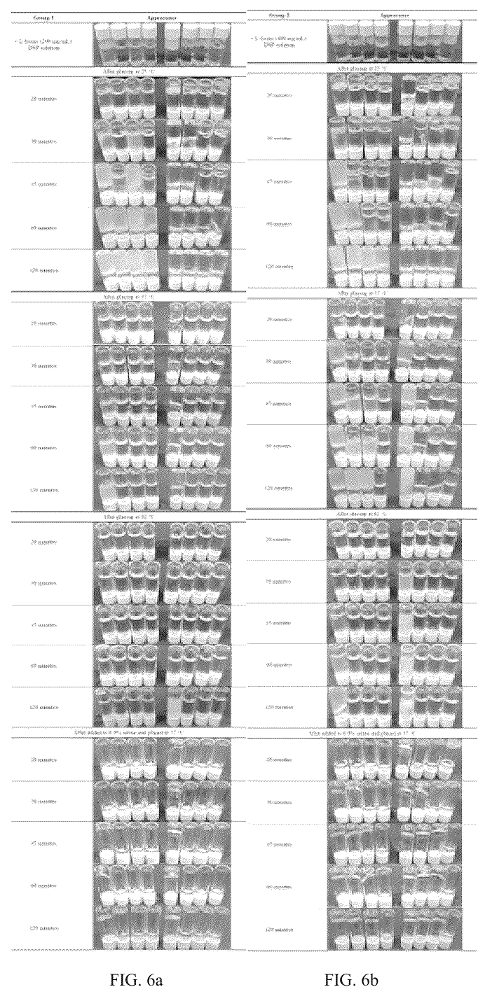

FIG. 6 showed that all groups formed transparent solution when L-lysine/DSP solutions were added to DCA-Na solutions. The pH value of mixed solutions ranged from 6.87-7.43 and 6.48-7.28 in 5% and 1% DCA-Na solution mixed with 200 mg/mL L-lysine solution at pH ranged from 4.0 to 7.0; 7.11-7.54 and 6.75-7.34 in 5% and 1% DCA-Na solution mixed with 400 mg/mL L-lysine solution at pH ranged from 4.0 to 7.0 (TABLE 10). At 200 mg/mL L-lysine test, mixtures of 5% DCA-Na and L-lysine/DSP added to 0.9% saline formed gel around 45 minutes at pH 4.0; mixtures of 1% DCA-Na and L-lysine/DSP added to 0.9% saline formed gel around 20 minutes at pH 4.0 (FIG. 6a). At 400 mg/mL L-lysine test, mixtures of 5% DCA-Na and L-lysine/DSP added to 0.9% saline formed gel around 30 minutes at pH 4.0 and 5.0, around 45 minutes at pH 6.0; mixtures of 1% DCA-Na and L-lysine/DSP added to 0.9% saline formed gel around 20 minutes at pH 4.0, around 30 minutes at pH 5.0 and 6.0 (FIG. 6b). This suggested that DCA-Na solutions can form gel after mixing with L-lysine DSP solutions and the time were shortened if concentration of L-lysine increased. Suitable pH for L-lysine/DSP solution is around pH 4.0-6.0.

In Example 5, the compositions added with 0.9% saline can form gel when the final concentration of DCA-Na was 8.123 or 40.615 mg/mL; the final concentration of lysine was 46.154 or 92.308 mg/mL; and the final concentration of DSP was 0.999 mg/mL. The compositions added with 0.9% saline can form gel when the final pH of the composition was 6.48-7.38.

### Example 6. Effects of DCA-Na and Lysine with DSP in Porcine Tissue

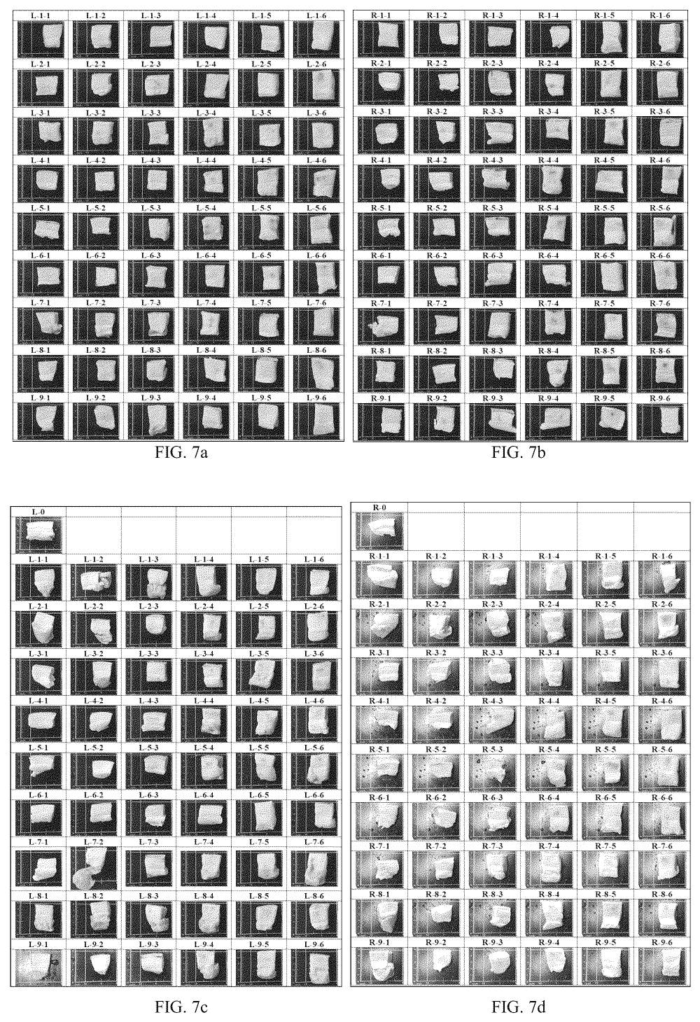

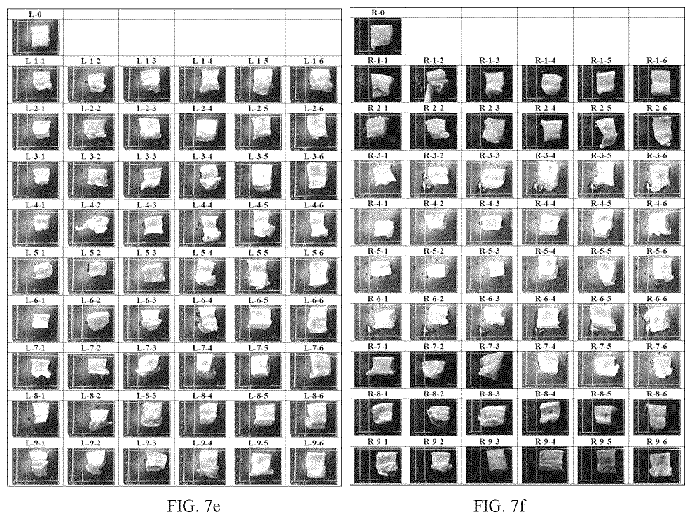

3 male pigs weighting at least 100 kg were anesthetized via intramuscular injection of 0.02 mg/kg Atropine and along with inhalation of 3% Isoflurane and 30-70% nitrous oxide (N₂O) mixed with oxygen (O₂). 0.5 mL L-lysine/DSP solution (pH 6.0) were added to 1 mL 1% or 5% DCA-Na solution and mixed until dissolved. Pigs were injected with 0.9% saline, 1% or 5% DCA-Na solutions with L-lysine/DSP solutions at different time points according to TABLE 11. Area for each injection site is 9 cm2 and compositions were injected at a depth of 0.5 cm at the center of each site. 54 sites were injected at each side of the pigs (Total 108 sites/pig). At day 0, animals were anesthetized via intramuscular injection of 0.02 mg/kg Atropine and 6 mg/kg Zoletil 50. Fat tissue samples were collected and cut in half from the center. Photos of tissues sections were recorded and shown in FIG. 7.

TABLE 11

| | | L-lysine/ | L-lysine/ | L-lysine/ | L-lysine/ |
|---|---|---|---|---|---|
| | L-lysine | DSP 0.1% | DSP 0.2% | DSP 0.4% | DSP 0.5% |
| | solution | solution | solution | solution | solution |
| L-lysine (g) | 1.5 | 1.5 | 1.5 | 1.5 | 1.5 |
| DSP (mg) | 0.0 | 3.0 | 6.0 | 12.0 | 24.0 |
| ddH₂O (mL) | 3.0 | 3.0 | 3.0 | 3.0 | 3.0 |
| Final | 500.0 | 500.0 | 500.0 | 500.0 | 500.0 |
| concentration | | | | | |
| of L-lysine | | | | | |
| (mg/mL) | | | | | |
| Final | 0.0 | 1.0 | 2.0 | 4.0 | 5.0 |
| concentration | | | | | |
| of DSP | | | | | |
| (mg/mL) | | | | | |

| | | | | | | | | Amount |
|---|---|---|---|---|---|---|---|---|
| | | | | | | | | of single |
| | | | | | | | | injection |
| Group | Composition | Time points for injection | | | | | | (uL) |
| 0 | Blank | — | | | | | | — |
| 1-1~1-6 | 0.9% Saline | 1-1 | 1-2 | 1-3 | 1-4 | 1-5 | 1-6 | 200 |
| | | −28 day | −21 day | −14 day | −7 day | −2 day | −3 hour | |
| 2-1~2-6 | 1% DCA-Na | 2-1 | 2-2 | 2-3 | 2-4 | 2-5 | 2-6 | 200 |
| | solution + | −28 day | −21 day | −14 day | −7 day | −2 day | −3 hour | |
| | Lysine HCl | | | | | | | |
| 3-1~3-6 | 1% DCA-Na | 3-1 | 3-2 | 3-3 | 3-4 | 3-5 | 3-6 | 400 |
| | solution + | −28 day | −21 day | −14 day | −7 day | −2 day | −3 hour | |
| | Lysine HCl | | | | | | | |
| 4-1~4-6 | 5% DCA-Na | 4-1 | 4-2 | 4-3 | 4-4 | 4-5 | 4-6 | 200 |
| | solution + | −28 day | −21 day | −14 day | −7 day | −2 day | ~3 hour | |
| | Lysine HCl | | | | | | | |
| 5-1~5-6 | 5% DCA-Na | 5-1 | 5-2 | 5-3 | 5-4 | 5-5 | 5-6 | 400 |
| | solution + | −28 day | −21 day | −14 day | −7 day | −2 day | −3 hour | |
| | Lysine HCl | | | | | | | |
| 6-1~6-6 | 5% DCA-Na | 6-1 | 6-2 | 6-3 | 6-4 | 6-5 | 6-6 | 200 |
| | solution + | −28 day | −21 day | −14 day | −7 day | −2 day | −3 hour | |
| | Lysine | | | | | | | |
| | HCl/DSP | | | | | | | |
| | 0.1% solution | | | | | | | |
| 7-1~7-6 | 5% DCA-Na | 7-1 | 7-2 | 7-3 | 7-4 | 7-5 | 7-6 | 200 |
| | solution + | −28 day | −21 day | −14 day | −7 day | −2 day | −3 hour | |
| | Lysine | | | | | | | |
| | HCl/DSP | | | | | | | |
| | 0.2% solution | | | | | | | |
| 8-1~8-6 | 5% DCA-Na | 8-1 | 8-2 | 8-3 | 8-4 | 8-5 | 8-6 | 200 |
| | solution + | −28 day | −21 day | −14 day | −7 day | −2 day | −3 hour | |
| | Lysine | | | | | | | |
| | HCl/DSP | | | | | | | |
| | 0.4% solution | | | | | | | |
| 9-1~9-6 | 5% DCA-Na | 9-1 | 9-2 | 9-3 | 9-4 | 9-5 | 9-6 | 200 |
| | solution + | −28 day | −21 day | ~14 day | −7 day | −2 day | −3 hour | |
| | Lysine | | | | | | | |
| | HCl/DSP | | | | | | | |
| | 0.8% solution | | | | | | | |

FIG. 7 showed that cytolysis occured at injected site when DCA-Na solutions were injected along with lysine or lysine/DSP at shallower depth. Increasing concentration or volume of DCA-Na resulted in stronger cytolytic reaction or inflammation as larger area of redness were observed (Groups 2-5). The cytolytic reaction or inflammation were much relieved after 7-14 days of injection, as less redness were observed. Increasing concentration of DSP also reduce the degree and area redness at injected site (Groups 6-9), suggesting that the additional of anti-inflammatory DSP could effectively reduce inflammation at injected site.

Compositions of DCA-Na and Lysine with DSP can effectively reduce fat, with less adverse effects, such as inflammation and redness.

### Example 7. Compositions of DCA-Na and Basic Amino Acids

To test if DCA-Na solutions and other basic, cationic amino acids form gel after mixing, DCA-Na solutions were mixed with acidic L-histidine (pH 5.0-5.2, Sigma-Aldrich) or L-arginine (pH 5.0-5.2, Sigma-Aldrich) solutions according to TABLES 12 and 13, respectively.

### Example 7.1. Compositions of DCA-Na and L-histidine

TABLE 12

| Group | 1 | 2 | 3 | 4 | 5 | 6 |
|---|---|---|---|---|---|---|
| L-Histidine (g) | 0.025 | 0.05 | 0.10 | 0.20 | 0.40 | 0.50 |
| ddH₂O (mL) | 10.0 | 10.0 | 10.0 | 10.0 | 10.0 | 10.0 |
| Final concentration of L-histidine | 2.5 | 5 | 10 | 20 | 40 | 50 |
| (mg/mL) | | | | | | |

| Group | 1-1 | 1-2 | 1-3 | 1-4 | 1-5 |
|---|---|---|---|---|---|
| 5% DCA-Na solution (mL) | 1.00 | 1.00 | 1.00 | 1.00 | 1.00 |
| 2.5 mg/mL L-histidine (mL) | 0.05 | 0.10 | 0.20 | 0.30 | 0.40 |
| (mg/mL) | | | | | |
| Final concentration of L-histidine | 0.12 | 0.23 | 0.42 | 0.58 | 0.71 |
| (mg/mL) | | | | | |
| Group | 1-6 | 1-7 | 1-8 | 1-9 | 1-10 |
| 1% DCA-Na solution (mL) | 1.00 | 1.00 | 1.00 | 1.00 | 1.00 |
| 2.5 mg/mL L-histidine (mL) | 0.05 | 0.10 | 0.20 | 0.30 | 0.40 |
| Final concentration of DCA-Na | 10.06 | 9.60 | 8.80 | 8.12 | 7.54 |
| (mg/mL) | | | | | |
| Final concentration of L-histidine | 0.12 | 0.23 | 0.42 | 0.58 | 0.71 |
| (mg/mL) | | | | | |
| Group | 2-1 | 2-2 | 2-3 | 2-4 | 2-5 |
| 5% DCA-Na solution (mL) | 1.00 | 1.00 | 1.00 | 1.00 | 1.00 |
| 5 mg/mL L-histidine (mL) | 0.05 | 0.10 | 0.20 | 0.30 | 0.40 |
| Final concentration of DCA-Na | 50.29 | 48.00 | 44.00 | 40.62 | 37.7 |
| (mg/mL) | | | | | |
| Final concentration of L-histidine | 0.24 | 0.45 | 0.83 | 1.15 | 1.43 |
| (mg/mL) | | | | | |
| Group | 2-6 | 2-7 | 2-8 | 2-9 | 2-10 |
| 1% DCA-Na solution (mL) | 1.00 | 1.00 | 1.00 | 1.00 | 1.00 |
| 5 mg/mL L-histidine (mL) | 0.05 | 0.10 | 0.20 | 0.30 | 0.40 |
| Final concentration of DCA-Na | 10.06 | 9.60 | 8.80 | 8.12 | 7.54 |
| (mg/mL) | | | | | |
| Final concentration of L-histidine | 0.24 | 0.45 | 0.83 | 1.15 | 1.43 |
| (mg/mL) | | | | | |
| Group | 3-1 | 3-2 | 3-3 | 3-4 | 3-5 |
| 5% DCA-Na solution (mL) | 1.00 | 1.00 | 1.00 | 1.00 | 1.00 |
| 10 mg/mL L-histidine (mL) | 0.05 | 0.10 | 0.20 | 0.30 | 0.40 |
| Final concentration of DCA-Na | 50.29 | 48.00 | 44.00 | 40.62 | 37.7 |
| (mg/mL) | | | | | |
| Final concentration of L-histidine | 0.48 | 0.91 | 1.67 | 2.31 | 2.86 |
| (mg/mL) | | | | | |
| Group | 3-6 | 3-7 | 3-8 | 3-9 | 3-10 |
| 1% DCA-Na solution (mL) | 1.00 | 1.00 | 1.00 | 1.00 | 1.00 |
| 10 mg/mL L-histidine (mL) | 0.05 | 0.10 | 0.20 | 0.30 | 0.40 |
| Final concentration of DCA-Na | 10.06 | 9.60 | 8.80 | 8.12 | 7.54 |
| (mg/mL) | | | | | |
| Final concentration of L-histidine | 0.48 | 0.91 | 1.67 | 2.31 | 2.86 |
| (mg/mL) | | | | | |
| Group | 4-1 | 4-2 | 4-3 | 4-4 | 4-5 |
| 5% DCA-Na solution (mL) | 1.00 | 1.00 | 1.00 | 1.00 | 1.00 |
| 20 mg/mL L-histidine (mL) | 0.05 | 0.10 | 0.20 | 0.30 | 0.40 |
| Final concentration of DCA-Na | 50.29 | 48.00 | 44.00 | 40.62 | 37.7 |
| (mg/mL) | | | | | |
| Final concentration of L-histidine | 0.95 | 1.82 | 3.33 | 4.62 | 5.71 |
| (mg/mL) | | | | | |
| Group | 4-6 | 4-7 | 4-8 | 4-9 | 4-10 |
| 1% DCA-Na solution (mL) | 1.00 | 1.00 | 1.00 | 1.00 | 1.00 |
| 20 mg/mL L-histidine (mL) | 0.05 | 0.10 | 0.20 | 0.30 | 0.40 |
| Final concentration of DCA-Na | 10.06 | 9.60 | 8.80 | 8.12 | 7.54 |
| (mg/mL) | | | | | |
| Final concentration of L-histidine | 0.95 | 1.82 | 3.33 | 4.62 | 5.71 |
| (mg/mL) | | | | | |
| Group | 5-1 | 5-2 | 5-3 | 5-4 | 5-5 |
| 5% DCA-Na solution (mL) | 1.00 | 1.00 | 1.00 | 1.00 | 1.00 |
| 40 mg/mL L-histidine (mL) | 0.05 | 0.10 | 0.20 | 0.30 | 0.40 |
| Final concentration of DCA-Na | 50.29 | 48.00 | 44.00 | 40.62 | 37.7 |
| (mg/mL) | | | | | |
| Final concentration of L-histidine | 1.90 | 3.64 | 6.67 | 9.23 | 11.43 |
| (mg/mL) | | | | | |
| Group | 5-6 | 5-7 | 5-8 | 5-9 | 5-10 |
| 1% DCA-Na solution (mL) | 1.00 | 1.00 | 1.00 | 1.00 | 1.00 |
| 40 mg/mL L-histidine (mL) | 0.05 | 0.10 | 0.20 | 0.30 | 0.40 |
| Final concentration of DCA-Na | 10.06 | 9.60 | 8.80 | 8.12 | 7.54 |
| (mg/mL) | | | | | |
| Final concentration of L-histidine | 1.90 | 3.64 | 6.67 | 9.23 | 11.43 |
| (mg/mL) | | | | | |
| Group | 6-1 | 6-2 | 6-3 | 6-4 | 6-5 |
| 5% DCA-Na solution (mL) | 1.00 | 1.00 | 1.00 | 1.00 | 1.00 |
| 50 mg/mL L-histidine (mL) | 0.05 | 0.10 | 0.20 | 0.30 | 0.40 |
| Final concentration of DCA-Na | 50.29 | 48.00 | 44.00 | 40.62 | 37.7 |
| (mg/mL) | | | | | |
| Final concentration of L-histidine | 2.38 | 4.55 | 8.33 | 11.54 | 14.29 |
| (mg/mL) | | | | | |
| Group | 6-6 | 6-7 | 6-8 | 6-9 | 6-10 |
| 1% DCA-Na solution (mL) | 1.00 | 1.00 | 1.00 | 1.00 | 1.00 |
| 50 mg/mL L-histidine (mL) | 0.05 | 0.10 | 0.20 | 0.30 | 0.40 |
| Final concentration of DCA-Na | 10.06 | 9.60 | 8.80 | 8.12 | 7.54 |
| (mg/mL) | | | | | |
| Final concentration of L-histidine | 2.38 | 4.55 | 8.33 | 11.54 | 14.29 |
| (mg/mL) | | | | | |

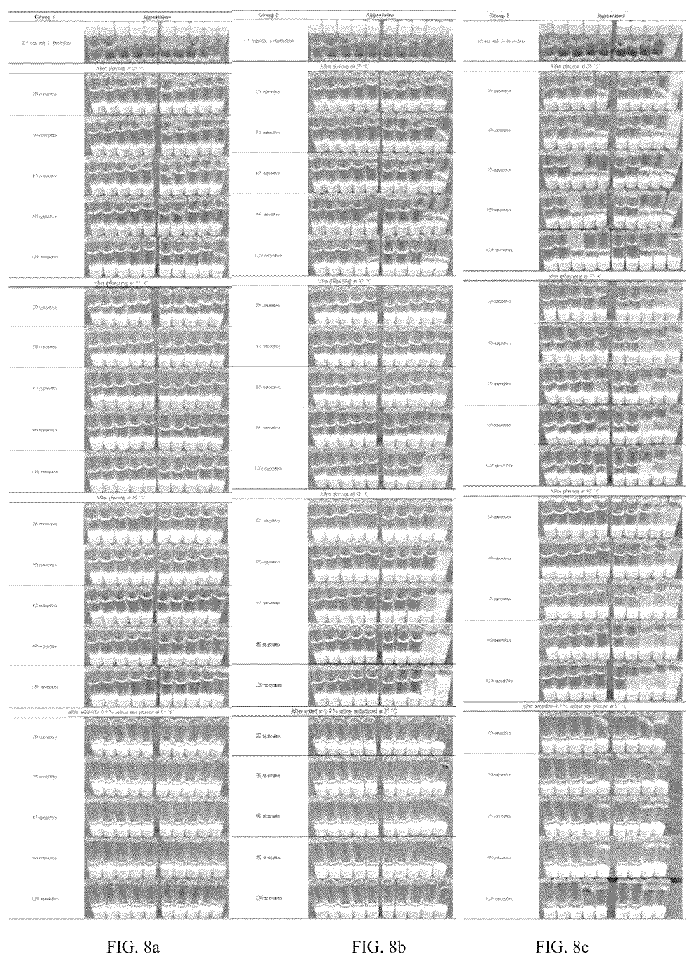

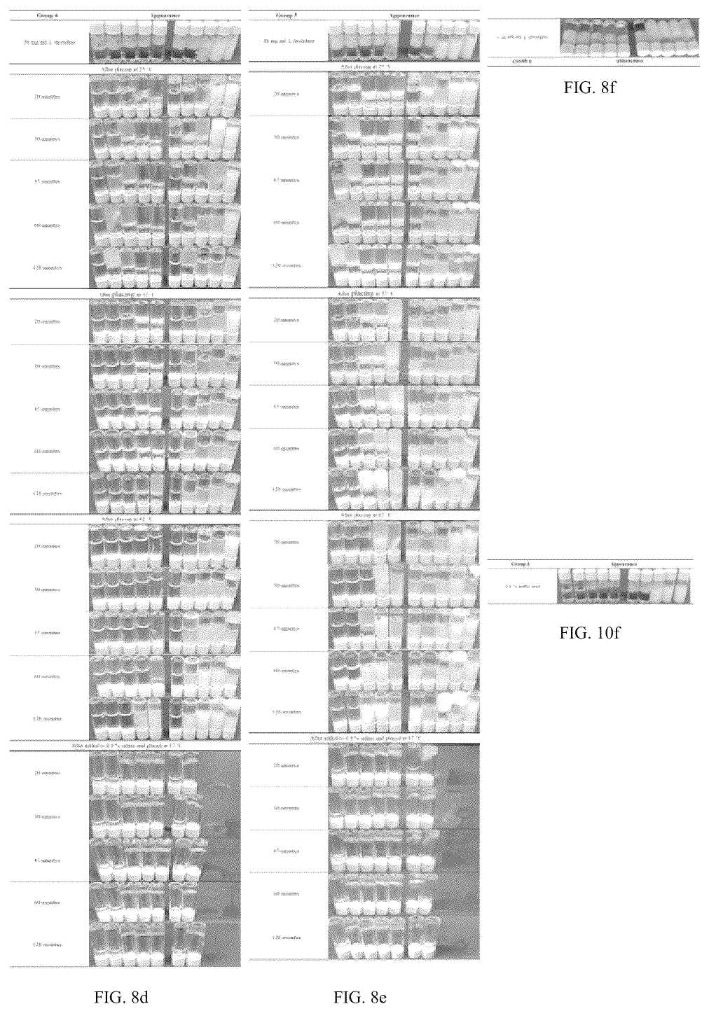

L-histidine solutions with concentration higher than 2.86 mg/mL precipitated after added to 1% DCA-Na solution (FIG. 8c-f); L-histidine solutions with concentration higher than 11.43 mg/mL precipitated after added to 5% DCA-Na solution (FIGS. 8e-f). Mixtures of DCA-Na and L-histidine with higher concentration of L-histidine started to form gel around 20 minutes when placed at 25° C., while mixtures placed at 37 or 42° C. formed suspensions (or precipitations) within a short period of time (FIG. 8b-f). Mixtures of 1% DCA-Na and L-histidine added to 0.9% saline formed gel around 20 minutes at L-histidine concentration ≥1.43 mg/mL (FIG. 8b-e). Mixtures of 5% DCA-Na and L-histidine added to 0.9% saline formed gel around 20 minutes at L-histidine concentration ≥2.86 mg/mL (FIG. 8c-e).

In Example 7.1, the compositions added with 0.9% saline can form gel when the final concentration of DCA-Na was 7.54-48.00 mg/mL, and the final concentration of L-histidine was 1.43˜11.43 mg/mL.

### Example 7.2. Compositions of DCA-Na and L-arginine

TABLE 13

| Group | 1 |
|---|---|
| L-arginine(g) | 5.0 |
| ddH₂O (mL) | 10.0 |
| Final concentration of L-arginine | 500 |
| (mg/mL) | |

| Group | 1-1 | 1-2 | 1-3 | 1-4 | 1-5 |
|---|---|---|---|---|---|
| 5% DCA-Na solution (mL) | 1.00 | 1.00 | 1.00 | 1.00 | 1.00 |
| 500 mg/mL L-arginine (mL) | 0.05 | 0.10 | 0.20 | 0.30 | 0.40 |
| Final concentration of DCA-Na | 50.29 | 48.00 | 44.00 | 40.62 | 37.7 |
| (mg/mL) | | | | | |
| Final concentration of L-arginine | 23.81 | 45.45 | 83.33 | 115.38 | 142.86 |
| (mg/mL) | | | | | |
| Group | 1-6 | 1-7 | 1-8 | 1-9 | 1-10 |
| 1% DCA-Na solution (mL) | 1.00 | 1.00 | 1.00 | 1.00 | 1.00 |
| 500 mg/mL L-arginine (mL) | 0.05 | 0.10 | 0.20 | 0.30 | 0.40 |
| Final concentration of DCA-Na | 10.06 | 9.60 | 8.80 | 8.12 | 7.54 |
| (mg/mL) | | | | | |
| Final concentration of L-arginine | 23.81 | 45.45 | 83.33 | 115.38 | 142.86 |
| (mg/mL) | | | | | |

*600 mg/mL L-arginine do not dissolve in ddH₂O.

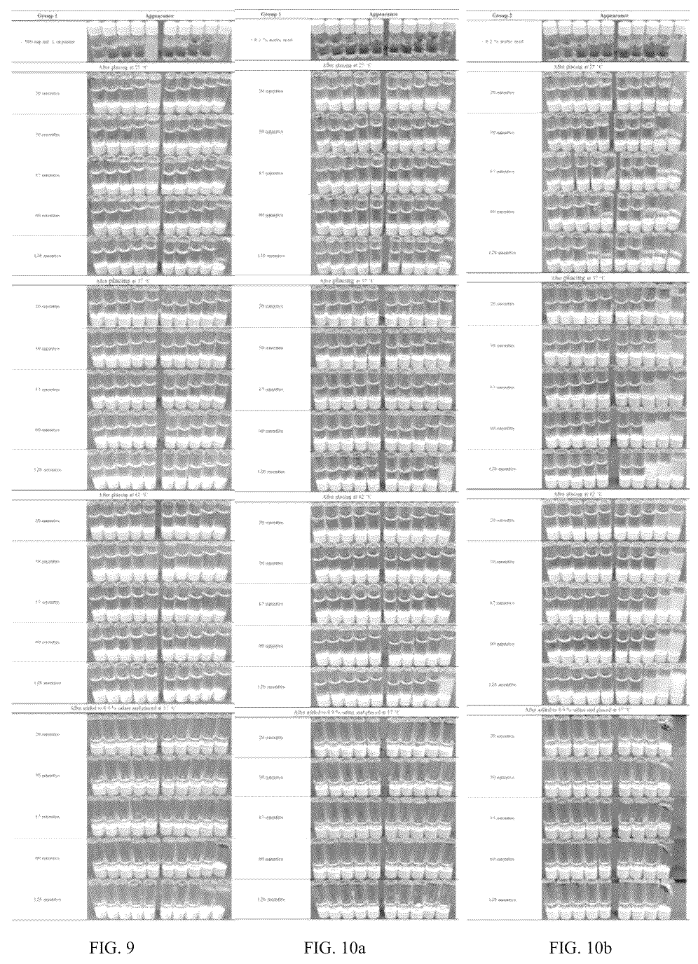

FIG. 9 showed that all groups formed transparent solution when 500 mg/mL L-arginine solutions were added to DCA-Na solutions. However, only group 1-10 formed gel around 60 minutes when placed at 25° C. and after added to 0.9% saline. All mixtures of DCA-Na and L-arginine placed at 37 or 42° C. did not formed gel in all tested time.

In Example 7.2, the compositions added with 0.9% saline can form gel when the final concentration of DCA-Na was 7.54 or 8.12 mg/mL, and the final concentration of L-arginine was 115.38 or 142.86 mg/mL.

These results revealed that although L-lysine, L-histidine and L-arginine belong to basic amino acids, the concentrations required to form gel were different. For instance, only high concentration of L-arginine and low concentration of DCA-Na formed gel and took longer time compared to L-lysine and L-histidine. On the other hand, low concentration of L-histidine was sufficient to form gel. In terms of forming gel compositions with DCA-Na, lysine may be the best, followed by histidine and arginine the worst.

### Example 8. Compositions of DCA-Na and Organic Acid

We have shown that pH value of solutions mixed with DCA-Na solutions affect the ability to form gel. To test if DCA-Na solutions and organic acid form gel after mixing, DCA-Na solutions were mixed with diluted acetic acid (Scharlau, Barcelona, Spain) according to TABLE 14.

TABLE 14

| Group | 1 | 2 | 3 | 4 | 5 | 6 |
|---|---|---|---|---|---|---|
| Acetic acid (mL) | 0.01 | 0.02 | 0.03 | 0.04 | 0.05 | 0.06 |
| ddH₂O) (mL) | 10.0 | 10.0 | 10.0 | 10.0 | 10.0 | 10.0 |
| Final concentration of acetic acid (%) | 0.1 | 0.2 | 0.3 | 0.4 | 0.5 | 0.6 |

| Group | 1-1 | 1-2 | 1-3 | 1-4 | 1-5 |
|---|---|---|---|---|---|
| 5% DCA-Na solution (mL) | 1.00 | 1.00 | 1.00 | 1.00 | 1.00 |
| 0.1% acetic acid (mL) | 0.05 | 0.10 | 0.20 | 0.30 | 0.40 |
| Final concentration of acetic acid | 4.76 | 9.09 | 16.67 | 23.08 | 28.57 |
| (x10⁻³%) | | | | | |
| Final concentration of DCA-Na | 50.29 | 48.00 | 44.00 | 40.62 | 37.7 |
| (mg/mL) | | | | | |
| Group | 1-6 | 1-7 | 1-8 | 1-9 | 1-10 |
| 1% DCA-Na solution (mL) | 1.00 | 1.00 | 1.00 | 1.00 | 1.00 |
| 0.1% acetic acid (mL) | 0.05 | 0.10 | 0.20 | 0.30 | 0.40 |
| Final concentration of DCA-Na | 10.06 | 9.60 | 8.80 | 8.12 | 7.54 |
| (mg/mL) | | | | | |
| Final concentration of acetic acid | 4.76 | 9.09 | 16.67 | 23.08 | 28.57 |
| (x10⁻³%) | | | | | |
| Group | 2-1 | 2-2 | 2-3 | 2-4 | 2-5 |
| 5% DCA-Na solution (mL) | 1.00 | 1.00 | 1.00 | 1.00 | 1.00 |
| 0.2% acetic acid (mL) | 0.05 | 0.10 | 0.20 | 0.30 | 0.40 |
| Final concentration of DCA-Na | 50.29 | 48.00 | 44.00 | 40.62 | 37.7 |
| (mg/mL) | | | | | |
| Final concentration of acetic acid | 9.52 | 18.18 | 33.33 | 46.15 | 57.14 |
| (x10⁻³%) | | | | | |
| Group | 2-6 | 2-7 | 2-8 | 2-9 | 2-10 |
| 1% DCA-Na solution (mL) | 1.00 | 1.00 | 1.00 | 1.00 | 1.00 |
| 0.2% acetic acid (mL) | 0.05 | 0.10 | 0.20 | 0.30 | 0.40 |
| Final concentration of DCA-Na | 10.06 | 9.60 | 8.80 | 8.12 | 7.54 |
| (mg/mL) | | | | | |
| Final concentration of acetic acid | 9.52 | 18.18 | 33.33 | 46.15 | 57.14 |
| (x10⁻³%) | | | | | |
| Group | 3-1 | 3-2 | 3-3 | 3-4 | 3-5 |
| 5% DCA-Na solution (mL) | 1.00 | 1.00 | 1.00 | 1.00 | 1.00 |
| 0.3% acetic acid (mL) | 0.05 | 0.10 | 0.20 | 0.30 | 0.40 |
| Final concentration of DCA-Na | 50.29 | 48.00 | 44.00 | 40.62 | 37.7 |
| (mg/mL) | | | | | |
| Final concentration of acetic acid | 14.29 | 27.27 | 50.00 | 69.23 | 85.71 |
| (x10⁻³%) | | | | | |
| Group | 3-6 | 3-7 | 3-8 | 3-9 | 3-10 |
| 1% DCA-Na solution (mL) | 1.00 | 1.00 | 1.00 | 1.00 | 1.00 |
| 0.3% acetic acid (mL) | 0.05 | 0.10 | 0.20 | 0.30 | 0.40 |
| Final concentration of DCA-Na | 10.06 | 9.60 | 8.80 | 8.12 | 7.54 |
| (mg/mL) | | | | | |
| Final concentration of acetic acid | 14.29 | 27.27 | 50.00 | 69.23 | 85.71 |
| (x10⁻³%) | | | | | |
| Group | 4-1 | 4-2 | 4-3 | 4-4 | 4-5 |
| 5% DCA-Na solution (mL) | 1.00 | 1.00 | 1.00 | 1.00 | 1.00 |
| 0.4% acetic acid (mL) | 0.05 | 0.10 | 0.20 | 0.30 | 0.40 |
| Final concentration of DCA-Na | 50.29 | 48.00 | 44.00 | 40.62 | 37.7 |
| (mg/mL) | | | | | |
| Final concentration of acetic acid | 19.05 | 36.36 | 66.67 | 92.30 | 114.29 |
| (x10⁻³%) | | | | | |
| Group | 4-6 | 4-7 | 4-8 | 4-9 | 4-10 |
| 1% DCA-Na solution (mL) | 1.00 | 1.00 | 1.00 | 1.00 | 1.00 |
| 0.4% acetic acid (mL) | 0.05 | 0.10 | 0.20 | 0.30 | 0.40 |
| Final concentration of DCA-Na | 10.06 | 9.60 | 8.80 | 8.12 | 7.54 |
| (mg/mL) | | | | | |
| Final concentration of acetic acid | 19.05 | 36.36 | 66.67 | 92.30 | 114.29 |
| (x10⁻³%) | | | | | |
| Group | 5-1 | 5-2 | 5-3 | 5.4 | 5-5 |
| 5% DCA-Na solution (mL) | 1.00 | 1.00 | 1.00 | 1.00 | 1.00 |
| 0.5% acetic acid (mL) | 0.05 | 0.10 | 0.20 | 0.30 | 0.40 |
| Final concentration of DCA-Na | 50.29 | 48.00 | 44.00 | 40.62 | 37.7 |
| (mg/mL) | | | | | |
| Final concentration of acetic acid | 23.81 | 45.45 | 83.33 | 115.38 | 142.86 |
| (x10⁻³%) | | | | | |
| Group | 5-6 | 5-7 | 5-8 | 5-9 | 5-10 |
| 1% DCA-Na solution (mL) | 1.00 | 1.00 | 1.00 | 1.00 | 1.00 |
| 0.5% acetic acid (mL) | 0.05 | 0.10 | 0.20 | 0.30 | 0.40 |
| Final concentration of DCA-Na | 10.06 | 9.60 | 8.80 | 8.12 | 7.54 |
| (mg/mL) | | | | | |
| Final concentration of acetic acid | 23.81 | 45.45 | 83.33 | 115.38 | 142.86 |
| (x10⁻³%) | | | | | |
| Group | 6-1 | 6-2 | 6-3 | 6-4 | 6-5 |
| 5% DCA-Na solution (mL) | 1.00 | 1.00 | 1.00 | 1.00 | 1.00 |
| 0.6% acetic acid (mL) | 0.05 | 0.10 | 0.20 | 0.30 | 0.40 |
| Final concentration of DCA-Na | 50.29 | 48.00 | 44.00 | 40.62 | 37.7 |
| (mg/mL) | | | | | |
| Final concentration of acetic acid | 28.57 | 54.55 | 100.00 | 138.46 | 171.43 |
| (x10⁻³%) | | | | | |
| Group | 6-6 | 6-7 | 6-8 | 6-9 | 6-10 |
| 1% DCA-Na solution (mL) | 1.00 | 1.00 | 1.00 | 1.00 | 1.00 |
| 0.6% acetic acid (mL) | 0.05 | 0.10 | 0.20 | 0.30 | 0.40 |
| Final concentration of DCA-Na | 10.06 | 9.60 | 8.80 | 8.12 | 7.54 |
| (mg/mL) | | | | | |
| Final concentration of acetic acid | 28.57 | 54.55 | 100.00 | 138.46 | 171.43 |
| (x10⁻³%) | | | | | |

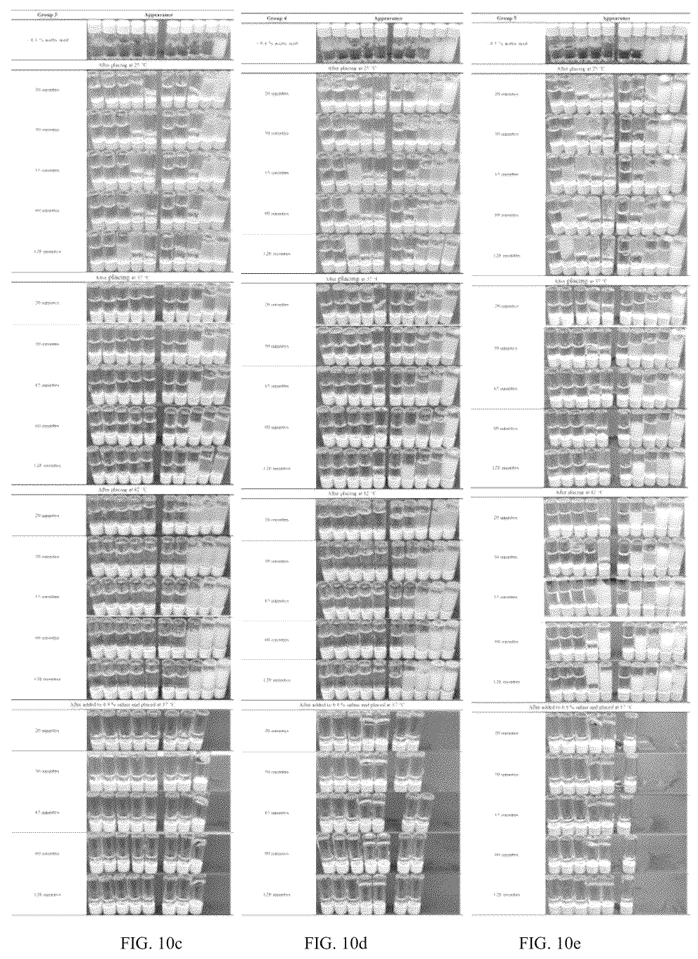

Acetic acid solutions with concentration higher than 57.14×10⁻³% precipitated after added to 1% DCA-Na solution (≤8.80 mg/mL); acetic acid solutions with concentration higher than 45.45×10⁻³% precipitated after added to 1% DCA-Na solution (≥9.60 mg/mL) (FIG. 10b-f). Acetic acid solutions with concentration higher than 171.43× 10⁻³% precipitated after added to 5% DCA-Na solution (≤37.7 mg/mL); acetic acid solutions with concentration higher than 100.00× 10⁻³% precipitated after added to 5% DCA-Na solution (≥44.00 mg/mL) (FIG. 10d-f). Mixtures of DCA-Na and acetic acid with higher concentration of acetic acid started to form gel around 20 minutes when placed at 25° C., while mixtures placed at 37 or 42° C. formed suspensions (or precipitations) within a short period of time (FIG. 10b-f). Mixtures of 1% DCA-Na and acetic acid added to 0.9% saline formed gel around 20 minutes at acetic acid concentration ≥46.15×10⁻³% (FIG. 10b-e). Mixtures of 5% DCA-Na and acetic acid added to 0.9% saline formed gel around 20 minutes at acetic acid concentration ≥92.30×10⁻³% (FIG. 10d-e).

In Example 8, the compositions added with 0.9% saline can form gel when the final concentration of DCA-Na was 7.54-40.62 mg/mL, and the final concentration of acetic acid was 46.15-142.86×10⁻³%.

The present invention demonstrated that cytolytic compound, especially deoxycholic acid, or its salt DCA-Na could form a slow-releasing gel, gel-forming solution or gel-forming suspension after mixing with amino acid (or cationic ion) at low pH or organic acid. Additional non-inflammatory drugs, such as lysine aspirin and dexamethasone sodium phosphate, and local anesthetic lidocaine could be added to the formulation of DCA-Na gel to reduce local inflammation. The present invention provides compositions of slow-releasing cytolytic compound, such as deoxycholic acid or its salt in gel or gel-forming solution (or suspension) for reduction of fat and with the addition of anti-inflammatory drugs and/or local anesthetic for the non-surgical reduction or removal of localized fat with reduced inflammation or other adverse effects and shorten the interval between each treatment and the whole treatment process. The injectable composition of the invention may optionally comprise saline, and may be in the form of gel during or after injection.

1. An injectable composition comprising cytolytic compound in gel, gel-forming solution or
gel-forming suspension for reduction of fat, comprising:
a cytolytic compound as a first component; and
a pharmaceutically acceptable excipient.

2. The injectable composition of claim 1, wherein the cytolytic compound is deoxycholic acid or a salt thereof.

3. The injectable composition of claim 1, wherein the cytolytic compound is DCA-Na, and
the injectable composition further comprises a second component selected from one or more of a basic amino acid or an organic acid.

4. The injectable composition of claim 3, wherein the concentration of DCA-Na is 7-51 mg/mL.

5. The injectable composition of claim 3, wherein the basic amino acid is L-lysine.

6. The injectable composition of claim 5, wherein the concentration of L-lysine is 11-145 mg/mL.

7. The injectable composition of claim 5, wherein the pH of L-lysine before mixing is <8.0, and the pH of the injectable composition is 6.45-7.75.

8. The injectable composition of claim 3, wherein the injectable composition further comprises an anti-inflammatory drug as a third component.

9. The injectable composition of claim 8, wherein the anti-inflammatory drug is aspirin.

10. The injectable composition of claim 9, wherein the concentration of aspirin is 14-100 mg/mL.

11. The injectable composition of claim 3, wherein the injectable composition further comprises a local anesthetic as a fourth component.

12. The injectable composition of claim 11, wherein the local anesthetic is Lidocaine.

13. The injectable composition of claim 8, wherein the anti-inflammatory drug is Dexamethasone Sodium Phosphate (DSP).

14. The injectable composition of claim 3, wherein the basic amino acid is L-histidine.

15. The injectable composition of claim 14, wherein the concentration of L-histidine is 1.4-11.5 mg/mL.

16. The injectable composition of claim 3, wherein the basic amino acid is L-arginine.

17. The injectable composition of claim 16, wherein the concentration of L-arginine is 115-143 mg/mL.

18. The injectable composition of claim 3, wherein the organic acid is acetic acid.

19. The injectable composition of claim 18, wherein the concentration of acetic acid is 46-143×10⁻³%.

20. Use of the injectable composition of claim 1, for the reduction or removal of localized fat in a subject in need thereof, wherein the injectable composition is subcutaneously injected into a subcutaneous injection site of the subject.

21. The use of claim 20, wherein the subcutaneous injection site is the localized fat within face, chin, arm, waist, abdomen or thigh of the subject.

22. Use of the injectable composition of claim 1, for production of a medicine for the reduction or removal of localized fat.

23. A method for reducing or removing localized fat in a subject in need thereof, comprising subcutaneously injecting to the subject, an effective amount of the injectable composition of claim 1.

24. The method of claim 23, wherein the injectable composition is subcutaneously injected into the localized fat within face, chin, arm, waist, abdomen or thigh of the subject.
